SEQUENCE LISTING (33 sequences)

SEQ ID NO: 1 - DNA, 7440 bp, Human poliovirus 1
ttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcataatgggtgctcaggtttcatcacagaaagtgggcgcacatgaaaactcaaatagagcgtatggtggttctaccattaattacaccaccattaattattatagagattcagctagtaacgcggcttcgaaacaggacttctctcaagacccttccaagttcaccgagcccatcaaggatgtcctgataaaaacagccccaatgctaaactcgccaaacatagaggcttgcgggtatagcgatagagtactgcaattaacactgggaaactccactataaccacacaggaggcggctaattcagtagtcgcttatgggcgttggcctgaatatctgagggacagcgaagccaatccagtggaccagccgacagaaccagacgtcgctgcatgcaggttttatacgctagacaccgtgtcttggacgaaagagtcgcgagggtggtggtggaagttgcctgatgcactgagggacatgggactctttgggcaaaatatgtactaccactacctaggtaggtccgggtacaccgtgcatgtacagtgtaacgcctccaaattccaccagggggcactaggggtattcgccgtaccagagatgtgtctggccggggatagcaacaccactaccatgcacaccagctatcaaaatgccaatcctggcgagaaaggaggcactttcacgggtacgttcactcctgacaacaaccagacatcacctgcccgcaggttctgcccggtggattacctccttggaaatggcacgttgttggggaatgcctttgtgttcccgcaccagataataaacctacggaccaacaactgtgctacactggtactcccttacgtgaactccctctcgatagatagtatggtaaagcacaataattggggaattgcaatattaccattggccccattaaattttgctagtgagtcctccccagagattccaatcaccttgaccatagcccctatgtgctgtgagttcaatggattaagaaacatcaccctgccacgcttacagggcctgccggtcatgaacacccctggtagcaatcaatatcttactgcagacaacttccagtcaccgtgtgcgctgcctgaatttgatgtgaccccacctattgacatacccggtgaagtaaagaacatgatggaattggcagaaatcgacaccatgattccctttgacttaagtgccacaaaaaagaacaccatggaaatgtatagggttcggttaagtgacaaaccacatacagacgatcccatactctgcctgtcactctctccagcttcagatcctaggttgtcacatactatgcttggagaaatcctaaattactacacacactgggcaggatccctgaagttcacgtttctgttctgtggattcatgatggcaactggcaaactgttggtgtcatacgcgcctcctggagccgacccaccaaagaagcgtaaggaggcgatgttgggaacacatgtgatctgggacataggactgcagtcctcatgtactatggtagtgccatggattagcaacaccacgtatcggcaaaccatagatgatagtttcaccgaaggcggatacatcagcgtcttctaccaaactagaatagtcgtccctctttcgacacccagagagatggacatccttggttttgtgtcagcgtgtaatgacttcagcgtgcgcttgttgcgagataccacacatatagagcaaaaagcgctagcacaggggttaggtcagatgcttgaaagcatgattgacaacacagtccgtgaaacggtgggggcggcaacatctagagacgctctcccaaacactgaagccagtggaccaacacactccaaggaaattccggcactcaccgcagtggaaactggggccacaaatccactagtcccttctgatacagtgcaaaccagacatgttgtacaacataggtcaaggtcagagtctagcatagagtctttcttcgcgcggggtgcatgcgtgaccattatgaccgtggataacccagcttccaccacgaataaggataagctatttgcagtgtggaagatcacttataaagatactgtccagttacggaggaaattggagttcttcacctattctagatttgatatggaacttacctttgtggttactgcaaatttcactgagactaacaatgggcatgccttaaatcaagtgtaccaaattatgtacgtaccaccaggcgctccagtgcccgagaaatgggacgactacacatggcaaacctcatcaaatccatcaatcttttacacctacggaacagctccagcccggatctcggtaccgtatgttggtatttcgaacgcctattcacacttttacgacggtttttccaaagtaccactgaaggaccagtcggcagcactaggtgactccctttatggtgcagcatctctaaatgacttcggtattttggctgttagagtagtcaatgatcacaacccgaccaaggtcacctccaaaatcagagtgtatctaaaacccaaacacatcagagtctggtgcccgcgtccaccgagggcagtggcgtactacggccctggagtggattacaaggatggtacgcttacacccctctccaccaaggatctgaccacatatggattcggacaccaaaacaaagcggtgtacactgcaggttacaaaatttgcaactaccacttggccactcaggatgatttgcaaaacgcagtgaacgtcatgtggagtagagacctcttagtcacagaatcaagagcccagggcaccgattcaatcgcaaggtgcaattgcaacgcaggggtgtactactgcgagtctagaaggaaatactacccagtatccttcgttggcccaacgttccagtacatggaggctaataactattacccagctaggtaccagtcccatatgctcattggccatggattcgcatctccaggggattgtggtggcatactcagatgtcaccacggggtgatagggatcattactgctggtggcgaagggttggttgcattttcagacattagagacttgtatgcctacgaagaagaagccatggaacaaggcatcaccaattacatagagtcacttggggccgcatttggaagtggatttactcagcagattagcgacaaaataacagagttgaccaatatggtgaccagtaccatcactgaaaagctacttaagaacttgatcaagatcatatcctcactagttattataactaggaactatgaagacaccacaacagtgctcgctaccctggcccttcttgggtgtgatgcttcaccatggcagtggcttagaaagaaagcatgcgatgttctggagataccttatgtcatcaagcaaggtgacagttggttgaagaagtttactgaagcatgcaacgcagctaagggactggagtgggtgtcaaacaaaatctcaaaattcattgattggctcaaggagaaaattatcccacaagctagagataagttggaatttgtaacaaaacttagacaactagaaatgctggaaaaccaaatctcaactatacaccaatcatgccctagtcaggaacaccaggaaattctattcaataatgtcagatggttatccatccagtctaagaggtttgcccctctttacgcagtggaagccaaaagaatacagaaactagagcatactattaacaactacatacagttcaagagcaaacaccgtattgaaccagtatgtttgctagtacatggcagccccggaacaggtaaatctgtagcaaccaacctgattgctagagccatagctgaaagagaaaacacgtccacgtactcgctacccccggatccatcacacttcgacggatacaaacaacagggagtggtgattatggacgacctgaatcaaaacccagatggtgcggacatgaagctgttctgtcagatggtatcaacagtggagtttataccacccatggcatccctggaggagaaaggaatcctgtttacttcaaattacgttctagcatccacaaactcaagcagaatttccccccccactgtggcacacagtgatgcattagccaggcgctttgcgttcgacatggacattcaggtcatgaatgagtattctagagatgggaaattgaacatggccatggctactgaaatgtgtaagaactgtcaccaaccagcaaactttaagagatgctgtcctttagtgtgtggtaaggcaattcaattaatggacaaatcttccagagttagatacagtattgaccagatcactacaatgattatcaatgagagaaacagaagatccaacattggcaattgtatggaggctttgtttcaaggaccactccagtataaagacttgaaaattgacatcaagacgagtccccctcctgaatgtatcaatgacttgctccaagcagttgactcccaggaggtgagagattactgtgagaagaagggttggatagtcaacatcaccagccaggttcaaacagaaaggaacatcaacagggcaatgacaattctacaagcggtgacaaccttcgccgcagtggctggagttgtctatgtcatgtataaactgtttgctggacaccagggagcatacactggtttaccaaacaaaaaacccaacgtgcccaccattcggacagcaaaggtacaaggaccagggttcgattacgcagtggctatggctaaaagaaacattgttacagcaactactagcaagggagagttcactatgttaggagtccacgacaacgtggctattttaccaacccacgcttcacctggtgaaagcattgtgatcgatggcaaagaagtggagatcttggatgccaaagcgctcgaagatcaagcaggaaccaatcttgaaatcactataatcactctaaagagaaatgaaaagttcagagacattagaccacatatacctactcaaatcactgagacaaatgatggagtcttgatcgtgaacactagcaagtaccccaatatgtatgttcctgtcggtgctgtgactgaacagggatatctaaatctcggtgggcgccaaactgctcgtactctaatgtacaactttccaaccagagcaggacagtgtggtggagtcatcacatgtactgggaaagtcatcgggatgcatgttggtgggaacggttcacacgggtttgcagcggccctgaagcgatcatacttcactcagagtcaaggtgaaatccagtggatgagaccttcgaaggaagtgggatatccaatcataaatgccccgtccaaaaccaagcttgaacccagtgctttccactatgtgtttgaaggggtgaaggaaccagcagtcctcactaaaaacgatcccaggcttaagacagactttgaggaggcaattttctccaagtacgtgggtaacaaaattactgaagtggatgagtacatgaaagaggcagtagaccactatgctggccagctcatgtcactagacatcaacacagaacaaatgtgcttggaggatgccatgtatggcactgatggtctagaagcacttgatttgtccaccagtgctggctacccttatgtagcaatgggaaagaagaagagagacatcttgaacaaacaaaccagagacactaaggaaatgcaaaaactgctcgacacatatggaatcaacctcccactggtgacttatgtaaaggatgaacttagatccaaaacaaaggttgagcaggggaaatccagattaattgaagcttctagtttgaatgactcagtggcaatgagaatggcttttgggaacctatatgctgcttttcacaaaaacccaggagtgataacaggttcagcagtggggtgcgatccagatttgttttggagcaaaattccggtattgatggaagagaagctgtttgcttttgactacacagggtatgatgcatctctcagccctgcttggttcgaggcactaaagatggtgcttgagaaaatcggattcggagacagagttgactacatcgactacctaaaccactcacaccacctgtacaagaataaaacatactgtgtcaagggcggtatgccatctggctgctcaggcacttcaatttttaactcaatgattaacaacttgattatcaggacactcttactgaaaacctacaagggcatagatttagaccacctaaaaatgattgcctatggtgatgatgtaattgcttcctacccccatgaagttgacgctagtctcctagcccaatcaggaaaagactatggactaactatgactccagctgacaaatcagctacatttgaaacagtcacatgggagaatgtaacattcttgaagagattcttcagggcagacgagaaatacccatttcttattcatccagtaatgccaatgaaggaaattcatgaatcaattagatggactaaagatcctaggaacactcaggatcacgttcgctctctgtgccttttagcttggcacaatggcgaagaagaatataacaaattcctagctaaaatcaggagtgtgccaattggaagagctttattgctcccagagtactcaacattgtaccgccgttggcttgactcattttagtaaccctacctcagtcgaattggattgggtcatactgttgtaggggtaaatttttctttaattcggag

SEQ ID NO: 2 - DNA, 7441 bp, Human poliovirus 1
ttaaaacagctctggggttgcacccgccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgttgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtctaggctgcgttggcggcctacctatggctaacgccatgggacgctagttgtgaacaaggtgtgaagagcctattgagctacataagaatcctccggcccctgaatgcggctaatcccaacctcggggcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagattcattatctatctgtttgctggattcgctccattgagtgtgtttactctaagtacaatttcaacagttatttcaatcagacaattgtatcataatgggtgctcaggtttcatcacagaaagtgggcgcacatgaaaactcaaatagagcgtatggtggttctaccattaattacaccaccattaattattatagagattcagctagtaacgcggcttcgaaacaggacttctctcaagacccttccaagttcaccgagcccatcaaggatgtcctgataaaaacatccccaatgctaaactcgccaaacatagaggcttgcgggtatagcgatagagtactgcaattaacactgggaaactccactataaccacacaggaggcggctaattcagtagtcgcttatgggcgttggcctgaatatctgagggacagcgaagccaatccagtggaccagccgacagaaccagacgtcgctgcatgcaggttttatacgctagacaccgtgtcttggacgaaagagtcgcgagggtggtggtggaagttgcctgatgcactgcgggacatgggactctttggccaaaatatgtactaccactacctaggtaggtccgggtacaccgtgcatgtacagtgtaacgcctccaaattccaccagggggcactaggggtattcgccgtaccagagatgtgtctggccggggatagcaacaccactaccatgcacaccagctatcaaaatgccaatcctggcgagaaaggaggcactttcacgggtacgttcactcctgacgacaaccagacatcacctgcccgtaggttctgcccggtggattacctctttggaaatggcacgttattggggaatgcctttgtgttcccgcaccagataataaacctacggaccaacaactgtgctacactggtactcccttacgtgaactccctctcgatagatagtatggtaaagcacaataattggggaattgcaatattaccattggccccattaaattttgctagtgagtcctccccagagattccaatcaccttgaccatagcccctatgtgctgtgagttcaatggattaagaaacattaccctgccacgcttacagggcctgccggtcatgaacacccctggtagcaatcaatatcttactgcagacaacttccagtcaccgtgtgcgctgcctgaatttgatgtgaccccacctattgacatacccggtgaagttaagaacatgatggaattggcagaaatcgacaccatgattccctttgacttaagtgcaaaaaaaaagaacaccatggaaatgtatagggttcggttaagtgacaaaccacatacagacgatcccatactctgcctgtcactctctccagcttcagatcctaggttgtcacatactatgcttggagaaatcctaaattactacacacactgggcaggatccctgaagttcacgtttctgttctgtggatccatgatggcaactggcaaactgttggtgtcatacgcgcctcctggagccgacccaccaaagaagcgtaaggaggcgatgttgggaacacatgtgatctgggacataggactgcagtcctcatgtactatggtagtgccatggattagcaacaccacgtatcggcaaaccatagatgatagtttcaccgaaggcggatacatcagcgtcttctaccaaaccagaatagtcgtccctctttcgacacccagagagatggacatccttggttttgtgtcagcgtgtaatgacttcagcgtgcgcttgatgcgagataccacacatatagagcaaaaagcgctagcacaggggttaggtcagatgcttgaaagcatgattgacaacacagtccgtgaaacggtgggggcggcaacgtctagagacgctctcccaaacactgaagccagtggaccagcacactccaaggaaattccggcactcaccgcagtggaaactggggccacaaatccactagtcccttctgatacagtgcaaaccagacatgttgtacaacataggtcaaggtcagagtctagcatagagtctttcttcgcgcggggtgcatgcgtggccattataaccgtggataactcagcttccaccaagaataaggataagctatttacagtgtggaagatcacttataaagatactgtccagttacggaggaaattggagttcttcacctattctagatttgatatggaatttacctttgtggttactgcaaatttcactgagactaacaatgggcatgccttaaatcaagtgtaccaaattatgtacgtaccaccaggcgctccagtgcccgagaaatgggacgactacacatggcaaacctcatcaaatccatcaatcttttacacctacggaacagctccagcccggatctcggtaccgtatgttggtatttcgaacgcctattcacacttttacgacggtttttccaaagtaccactgaaggaccagtcggcagcactaggtgactccctctatggtgcagcatctctaaatgacttcggtattttggctgttagagtagtcaatgatcacaacccgaccaaggtcacctccaaaatcagagtgtatctaaaacccaaacacatcagagtctggtgcccgcgtccaccgagggcagtggcgtactacggccctggagtggattacaaggatggtacgcttacacccctctccaccaaggatctgaccacatatggattcggacaccaaaacaaagcggtgtacactgcaggttacaaaatttgcaactaccatttggccactcaggaagatttgcaaaacgcagtgaacgtcatgtggaatagagacctcttagtcacagaatcaagagcccagggcaccgattcaatcgcaaggtgcaattgcaacgcaggggtgtactactgcgagtctagaaggaaatactacccagtatccttcgttggcccaacgttccagtacatggaggctaataactattacccagctaggtaccagtcccatatgctcattggccatggattcgcatctccaggggattgtggtggcatactcagatgtcaccacggggtgatagggatcattactgctggtggagaagggttggttgcatttacagacattagagacttgtatgcctacgaagaagaagccatggaacaaggcatcaccaattacatagagtcacttggggccgcatttggaagtggatttactcagcagattggagacaaaataacagagttgactaatatggtgaccagtaccatcactgaaaagctacttaagaacttgatcaagatcatatcctcactagttattataactaggaattatgaagacaccacaacagtgctcgctaccctggcccttcttgggtgtgatgcttcaccatggcagtggcttagaaagaaagcatgcgatgttctggagataccttatgtcaccaagcaaggtgacagttggttgaagaagtttactgaagcatgcaacgcagctaagggactggagtgggtgtcaaacaaaatctcaaaattcattgattggctcaaggagaaaattatcccacaagctagagataagttggaatttgtaacaaaacttagacaactagaaatgctggaaaaccaaatctcaactatacaccaatcatgccctagtcaggaacaccaggaaattctattcaataatgtcagatggttatccatccagtctaagaggtttgcccctctttacgcagtggaagccaaaagaatacagaaactagagcataccattaacaactacatacagttcaagagcaaacaccgtattgaaccagtatgtttgctagtacatggcagccccggaacaggtaaatctgtagcaaccaacctgattgctagagccatagctgaaagagaaaacacgtccacgtactcgctacccccggatccatcacacttcgacggatacaaacaacagggagtggtgattatggacgacctgaatcaaaacccagatggtgcggacatgaagctgttctgtcagatggtatcaacagtggagtttataccacccatggcatccctggaggagaaaggaatcctgtttacttcaaattacgttctagcatccacgaactcaagcagaatttccccccccactgtggcacacagtgatgcattagccaggcgctttgcgttcgacatggacattcaggtcatgaatgagtattctagagatgggaaattgaacatggccatggctactgaaatgtgtaagaactgtcaccaaccagcaaactttaagagatgctgtcctttagtgtgtggtaaggcaattcaattaatggacaaatcttccagagttagatacagtattgaccagatcactacaatgattatcaatgagagaaacagaagatccaacattggcaattgtatggaggctttgttccaaggaccactccagtataaagacttgaagattgacatcaagacgagtccccctcctgaatgtatcaatgacttgctccaagcagttgactcccaggaggtgagagattactgtgagaagaagggttggatagtcaacatcaccagccaggttcaaacagaaaggaacatcaacagggcaatgacaattctacaagcggtgacaaccttcgccgcagtggctggagttgtctatgtcatgtataaactgtttgctggacaccagggagcatacactggtttaccaaacaaaaaacccaacgtgcccaccattaggacagcaaaggtacaagggccagggttcgattacgcagtggctatggctaaaagaaacattgttacagcaactactagcaagggagagttcactatgttaggagtccacgacaacgtggctattttaccaacccacgcttcacctggtgaaagcattgtgatcgatggcaaagaagtggagatcttggatgccaaagcgctcgaagatcaagcaggaaccaatcttgaaatcactataatcactctaaagagaaatgaaaagttcagagacattagaccacatatacctactcaaatcactgagacaaatgatggagtcttgatcgtgaacactagcaagtaccccaatatgtatgttcctgtcggtgctgtgactgaacagggatatctaaatctcggtgggcgccaaactgctcgtactctaatgtacaactttccaaccagagcaggacagtgtggtggagtcatcacatgtactgggaaagtcatcgggatgcatgttggtgggaacggttcacacgggtttgcagcggccctgaagcgatcatacttcactcagagtcaaggtgaaatccagtggatgagaccttcgaaggaagtgggatatccaatcataaatgccccgtccaaaaccaagcttgaacccagtgctttccactatgtgtttgaaggggtgaaggaaccagcagtcctcactaaaaacgatcccaggcttaagacaaactttgaggaggcaattttctccaagtacgtgggtaacaaaattactgaagtggatgagcacatgaaagaggcagtagaccactatgctggccagctcatgtcactagacatcaacacagaacaaatgtgcttggaggatgccatgtatggcactgatggtctagaagcacttgatttgtccaccagtgctggctacccttatgtagcaatgggaaagaagaagagagatatcttgaacaaacaaaccagagacactaaggaaatgcaaaaactgctcgacacatatggaatcaacctcccactggtgacttatgtaaaggatgaacttagatccaaaacaaaggttgagcaggggaaatccagattaattgaagcttctagtttgaatgactcagtggcaatgagaatggcttttgggaacctatatgctgcttttcacaaaaacccaggagtgataacaggttcagcagtagggtgcgatccagatttgttttggagcaaaattccggtattgatggaagagaagctgtttgcctttgactacacagggtatgatgcatctctcagccctgcttggttcgaggcactagagatggtgcttgagaaaatcggattcggagacagagttgactacatcgactacctaaaccactcacaccacctgtacaagaataaaacatactgtgtcaagggcggtatgccatctggttgctcaggcacttcaatttttaactcaatgattaacaacttgattatcaggacactcttactgaaaacctacaagggcatagatttagaccacctaaaaatgattgcctatggtgatgatgtaattgcttcctacccccatgaagttgacgctagtctcctagcccaatcaggaaaagactatggactaactatgactccagctgacaaatcagctatatttgaaacagtcacatgggagaatgtaacattcttgaagagattcttcagggcagacgagaaatacccatttcttattcatccagtaatgccaatgaaggaaattcatgaatcaattagatggacaaaagatcctaggaacactcaggatcacgttcgctctctgtgcctattagcttggcacaatggcgaagaagaatataacaaattcctagctaaaatcaggagtgtgccaattggaagagctttattgctcccagagtactcaacattgtaccgccgttggcttgactcattttagtaaccctacctcagtcgaattggattgggtcatactgctgtaggggtaaatttttctttaattcggagg

SEQ ID NO: 3 - DNA, 20 bp, Artificial
  Forward primer (i)
ccctttgacttaagthccac

SEQ ID NO: 4 - DNA, 20 bp, Artificial
  Forward primer (ii)
ccctttgacttaagthcaaa

SEQ ID NO: 5 - DNA, 20 bp, Artificial
  Forward primer (iii)
ccatggtgttcttttvtgtg

SEQ ID NO: 6 - DNA, 20 bp, Artificial
  Forward primer (iv)
ccatggtgttcttttvtttt

SEQ ID NO: 7 - DNA, 20 bp, Artificial
  Forward primer (v)
gatttactcagcagavtagc

SEQ ID NO: 8 - DNA, 20 bp, Artificial
  Forward primer (vi)
gatttactcagcagavtgga

SEQ ID NO: 9 - DNA, 20 bp, Artificial
  Forward primer (vii)
aactctgttattttgvcgct

SEQ ID NO: 10 - DNA, 20 bp, Artificial
  Forward primer (viii)
aactctgttattttgvctcc

SEQ ID NO: 11 - DNA, 21 bp, Artificial
  Reverse primer (i)/(ii)
gatcctgcccagtgtgtgtag

SEQ ID NO: 12 - DNA, 22 bp, Artificial
  Forward primer I
ggttgttgagggagtcacgaga

SEQ ID NO: 13 - DNA, 22 bp, Artificial
  Reverse primer I
ccctgtctctacggctgttagc

SEQ ID NO: 14 - DNA, 17 bp, Artificial
  Forward primer II
gcaattacgccgcaagc

SEQ ID NO: 15 - DNA, 20 bp, Artificial
  Reverse primer II
gtgtaggtgctcctggaggt

SEQ ID NO: 16 - DNA, 24 bp, Artificial
  Forward primer III
aaggaattggtgacatgattgagg

SEQ ID NO: 17 - DNA, 18 bp, Artificial
  Reverse primer III
ctcggctttgtgtcaggc

SEQ ID NO: 18 - DNA, 24 bp, Artificial
  Forward primer IV
aatgaccagattggtgattccttg

SEQ ID NO: 19 - DNA, 25 bp, Artificial
  Reverse primer IV
gtaaatgcggactttggaggttact

SEQ ID NO: 20 - DNA, 29 bp, Artificial
  Polio 1 probe
agggttcggttaagtgacaaaccacatac

SEQ ID NO: 21 - DNA, 25 bp, Artificial
  MEF-1 probe
acaccactgacacctgccaacaact

SEQ ID NO: 22 - DNA, 25 bp, Artificial
  Saukett probe
ttcgtggtaacagccaacttcacca

SEQ ID NO: 23 - DNA, 22 bp, Artificial
  Sabin 2 probe
tggaagtcgggggaaccaatgc

SEQ ID NO: 24 - DNA, 28 bp, Artificial
  Sabin 3 probe
tgtgatcattgacaacacgaactgccaa

SEQ ID NO: 25 - DNA, 21 bp, Artificial
  Forward primer Sabin 1
tccctttgacttaagthcaaa

SEQ ID NO: 26 - DNA, 180 bp, Human poliovirus 1
gcagaaatcgacaccatgattccctttgacttaagtgcaaaaaaaaagaacaccatggaaatgtatagggttcggttaagtgacaaaccacatacagacgatcccatactctgcctgtcactctctccagcttcagatcctaggttgtcacatactatgcttggagaaatcctaaattac

SEQ ID NO: 27 - DNA, 179 bp, Human poliovirus 1
cagaaatcgacaccatgattccctttgacttaagtgccacaaaaaagaacaccatggaaatgtatagggttcggttaagtgacaaaccacatacagacgatcccatactctgcctgtcactctctccagcttcagatcctaggttgtcacatactatgcttggagaaatcctaaattac

SEQ ID NO: 28 - DNA, 100 bp, Human poliovirus 2
aaggaattggtgacatgattgagggggccgttgaagggattactaaaaatgcattggttcccccgacttccaccaatagcctgcctgacacaaagccgag

SEQ ID NO: 29 - DNA, 127 bp, Human poliovirus 2
ggttgttgagggagtcacgagaaatgccttgacaccactgacacctgccaacaacttgcctgatacacaatctagtggcccagcccactctaaggaaacaccagcgctaacagccgtagagacaggg

SEQ ID NO: 30 - DNA, 120 bp, Human poliovirus 3
aatgaccagattggtgattccttgtacagcgccatgacagttgatgactttggtgtattggcagttcgtgttgtcaatgatcacaaccccactaaagtaacctccaaagtccgcatttac

SEQ ID NO: 31 - DNA, 152 bp, Human poliovirus 3
gcaattacgccgcaagctggaattctttacatactctcgatttgacatggaattcaccttcgtggtaacagccaacttcaccaacaccaataacgggcatgcactgaatcaagtgtaccagataatgtacatacctccaggagcacctacac

SEQ ID NO: 32 - DNA, 20 bp, Artificial
  Forward primer (ix)
gatttactcagcagataggg

SEQ ID NO: 33 - DNA, 20 bp, Artificial
  Forward primer x
aactctgttattttgvcccc

(19) United States
(12) Patent Application Publication
(10) Pub. No.: US 2015/0031015 A1
(43) Pub. Date: Jan. 29, 2015

(54) IDENTIFICATION OF POLIOVIRUS STRAINS

(71) Applicant: De Staat der Nederlanden, Vert. Door De Minister Van VWS, Den Haag (NL)

(72) Inventors: Bernardus Metz, Nieuwer ter Aa (NL); Olaf Egidius Marie Nijst, Utrecht (NL); Justin Johannes Mouthaan, Utrecht (NL); Dirk Ronald Mekkes, Bilthoven (NL)

(73) Assignees: De Staat der Nederlanden, Vert. Door De Minister Van VWS, Ministerie van Volksgezondheid, Welzijn; en Sport, Den Haag (NL)

(21) Appl. No.: 14/364,594

(22) PCT Filed: Dec. 14, 2012
(86) PCT No.: PCT/NL2012/050887

§ 371 Date: Jun. 11, 2014

Related U.S. Application Data
(60) Provisional application No. 61/570,399, filed on Dec. 14, 2011.

(30) Foreign Application Priority Data
Dec. 14, 2011 (EP) 11193553.2

(51) Int. Cl. C12Q 1/70

(52) U.S. Cl. CPC C12Q 1/701; C12Q 2600/158
USPC 435/5; 536/24.32; 536/24.33

(57) ABSTRACT

The present invention relates to methods for identifying and/or distinguishing polioviral strains, in particular polioviral strains used in vaccine production. The methods are based on selective hybridisation with oligonucleotides, i.e. primers and/or probes, that allow to distinguish between closely related but different polioviral strains on the basis of nucleotidepolymorphisms existing between those polioviral strains. Preferably, the methods employ amplification or amplification-ligation assays for detecting the selective hybridisation. The invention further relates to oligonucleotides for use in the methods of the invention and kits comprising such oligonucleotides and optionally enzymes and buffers for carrying out the methods of the invention.

### FIELD OF THE INVENTION

The present invention relates to the fields of virology and viral vaccinology. In particular the present invention relates means and methods for identification of vaccine-specific poliovirus strains.

### BACKGROUND OF THE INVENTION

The use of live-attenuated oral polio vaccine (OPV) and inactivated polio vaccine (IPV) has drastically reduced the number of cases and outbreaks of poliomyelitis (Dutta, 2008; Crawford and Buttery, 2010). The Global Polio Eradication Initiative of the World Health Organization (WHO) is now focusing on the elimination of the disease and eradication of wild polio virus (WHO, 2003; Sutter et al., 2003). Despite considerable effort expanded in mass vaccinations, dozens of polio cases are still reported in the last years (Crawford and Buttery, 2010; Global Polio Eradication Initiative. Global polio case count. 2011. Available from: (accessed Nov. 14, 2011); Kew et al., 2005). Vaccination with OPV can cause the introduction of vaccine-derived polioviruses, resulting in outbreaks of poliomyelitis (Kew et al., 2005). Some of these vaccine-derived polioviruses are highly virulent and transmissible (Boot et al., 2004; Kew et al., 2005). IPV does obviously not have this disadvantage. However, the production capacity of IPV is currently very limited and increased IPV use will require building more production facilities. These facilities will have to deal with stringent arrangements in containment, since IPV is produced using virulent polio strains (2003). Therefore the WHO decided to stimulate the development of IPV based on attenuated polioviruses, e.g. the Sabin strains (Heymann et al., 2005; Heymann et al., 2006). Thus the risk of hazardous effects by virus escape from a production facility or accidental infection of manufacturing personnel can be minimised, although not completely eliminated (Kew et al., 2005).

During transition from IPV based on wild type strains to Sabin strains, producers of IPV might use both wild-type and attenuated poliovirus strains (Table 1). The unambiguous identification of poliovirus strains used for IPV production will be an important quality control test for vaccine release. Currently serological methods are used for the identification and quantification of the three poliovirus serotypes present in IPV, but in general they do not discriminate between wild-type and attenuated vaccine strains (Westdijk et al., 2011). Although serological assays are available that differentiate between wild-type and attenuated poliovirus strains, they require highly specific antisera (van der Avoort et al., 1995), the preparation of which is complex and laborious (van Wezel and Hazendonk, 1979). Alternatively, molecular methods are available for routine analysis of polioviruses in field isolates that can identify wild-type or attenuated poliovirus strains. The techniques are either based on nucleic acid hybridization (Kilpatrick et al., 1996) or reverse transcription PCR (Kilpatrick et al., 1998; Boot et al., 2004; Kilpatrick et al., 2004; Kilpatrick et al., 2009). Each technique has his own advantages and disadvantages (Table 2). However these methods described in the literature have to be modified before they can be used for the identification vaccine-specific poliovirus strains. There is therefore still a need in the art for methods and means that allow to rapidly and accurately identify and distinguish the various vaccine-specific poliovirus strains used in vaccine production, including e.g. Mahoney, MEF-1, Saukett H, Sabin type 1, Sabin type 2 and Sabin type 3.

### SUMMARY OF THE INVENTION

In a first aspect the invention relates to a method for the identification of a poliovirus strain in a sample, wherein the method comprises the step of selective hybridisation of an oligonucleotide to the polioviral nucleic acid in the sample, wherein the oligonucleotide is at least one of: a) an oligonucleotide comprising at least 12 contiguous nucleotides of SEQ ID NO: 1 or its complement and wherein the oligonucleotide comprises the sequence of positions 1942-1944 of SEQ ID NO: 1 or its complement at the 5′ or 3′ end; b) an oligonucleotide comprising at least 12 contiguous nucleotides of SEQ ID NO: 1 or its complement and wherein the oligonucleotide comprises the sequence of positions 3894-3896 of SEQ ID NO: 1 or its complement at the 5′ or 3′ end; c) an oligonucleotide comprising at least 12 contiguous nucleotides of SEQ ID NO: 2 or its complement and wherein the oligonucleotide comprises the sequence of positions 1942-1944 of SEQ ID NO: 2 or its complement at the 5′ or 3′ end; and, d) an oligonucleotide comprising at least 12 contiguous nucleotides of SEQ ID NO: 2 or its complement and wherein the oligonucleotide comprises the sequence of positions 3894-3896 of SEQ ID NO: 2 or its complement at the 5′ or 3′ end; whereby, selective hybridisation with the oligonucleotide in a) is indicative of the presence of a poliovirus strain selected from the group consisting of Mahoney type 1, Brunhilde, CHAT and Cox; selective hybridisation with the oligonucleotide in b) is indicative of the presence of the Mahoney type 1 poliovirus strain; selective hybridisation with the oligonucleotide in c) is indicative of the presence of the Sabin type 1 poliovirus strain; and, selective hybridisation with the oligonucleotide in d) is indicative of the presence of a poliovirus strain selected from the group consisting of Sabin type 1, CHAT and Cox. Preferably in the method, the oligonucleotide comprises a mismatch to both SEQ ID NO: 1 and 2, or their complements, more preferably, the mismatch is at a position corresponding to positions 1940, 1946, 3892 or 3898 of SEQ ID NO: 1. In one embodiment of the method according to invention, the selective hybridisation of the oligonucleotide is detected by an amplification or an amplification-ligation assay.

In a preferred embodiment of the invention, the method comprises the steps of: a) amplifying at least a portion of polioviral nucleic acid in the sample with a primer pair comprising a forward primer that is at least one of: (i) a forward primer comprising at least 12 contiguous nucleotides and the 3′-end of the sequence: 5′-CCCTTTGACTTAAGTHCCAC-3′, wherein H is a nucleotide that is incapable of base pairing with C; (ii) a forward primer comprising at least 12 contiguous nucleotides and the 3′-end of the sequence: 5′-CCCTTTGACTTAAGTHCAAA-3′, wherein H is a nucleotide that is incapable of base pairing with C; (iii) a forward primer comprising at least 12 contiguous nucleotides and the 3′-end of the sequence: 5′-CCATGGTGTTCTTTTVTGTG-3′, wherein V is a nucleotide that is incapable of base pairing with A; (iv) a forward primer comprising at least 12 contiguous nucleotides and the 3′-end of the sequence: 5′-CCATGGTGTTCTTTTVTTTT-3′, wherein V is a nucleotide that is incapable of base pairing with A; (v) a forward primer comprising at least 12 contiguous nucleotides and the 3′-end of the sequence: 5′-GATTTACTCAGCAGAVTAGC-3′, wherein V is a nucleotide that is incapable of base pairing with A; (vi) a forward primer comprising at least 12 contiguous nucleotides and the 3′-end of the sequence: 5′-GATTTACTCAGCAGAVTGGA-3′, wherein V is a nucleotide that is incapable of base pairing with A; (vii) a forward primer comprising at least 12 contiguous nucleotides and the 3′-end of the sequence: 5′-AACTCTGTTATTTTGVCGCT-3′, wherein V is a nucleotide that is incapable of base pairing with A; and, (viii) a forward primer comprising at least 12 contiguous nucleotides and the 3′-end of the sequence: 5′-AACTCTGTTATTTTGVCTCC-3′, wherein V is a nucleotide that is incapable of base pairing with A; and a reverse primer, whereby a reverse primer in a pair with a forward primer produces an amplicon with the forward primers (i), (iii), (v) and (vii) on a reference cDNA template comprising the sequence of a Mahoney poliovirus strain or with the forward primer (ii), (iv), (vi) and (viii) on a reference cDNA template comprising the sequence of a Sabin type 1 poliovirus strain; and, b) detecting whether an amplicon is obtained in step a), whereby an amplicon produced with at least one of forward primers (i), (iii), (v) and (vii) is indicative of the presence of a poliovirus strain selected from the group consisting of Mahoney, Brunhilde, CHAT and Cox; and, whereby an amplicon produced with at least one of forward primer (ii) (iv) (vi) and (viii) is indicative of the presence of the Sabin type 1 poliovirus strain. Preferably in this embodiment of the method, the reverse primer comprises at its 3′-end a sequence of at least 14 contiguous nucleotides that are complementary to a sequence in an elongation product obtained on a polioviral template with a forward primer defined above. Preferably, the forward primer that is at least one of: (i) a forward primer comprising the sequence: 5′-CCCTTTGACTTAAGTHCCAC-3′, wherein H is A, C, T or U; and, (ii) a forward primer comprising the sequence: 5′-CCCTTTGACTTAAGTHCAAA-3′, wherein H is A, C, T or U; and the reverse primer is 5′-GATCCTGCCCAGTGTGTGTAG-3′. Alternatively or in addition, the method may comprise the steps of: d) amplifying at least a portion of polioviral nucleic acid in the sample with a primer pair comprising a forward primer that is at least one of: (ix) a forward primer comprising at least 12 contiguous nucleotides and the 3′-end of the sequence: 5′-GATTTACTCAGCAGATAGGG-3′ (SEQ ID NO: 32); (x) a forward primer comprising at least 12 contiguous nucleotides and the 3′-end of the sequence: 5′-AACTCTGTTATTTTGVCCCC-3′ (SEQ ID NO: 33), wherein V is a nucleotide that is incapable of base pairing with A; and a reverse primer, whereby a reverse primer in a pair with a forward primer produces an amplicon with the forward primers (ix) and (x) on a reference cDNA template comprising the sequence of the Brunhilde poliovirus strain; and, e), detecting whether an amplicon is obtained in step d), whereby an amplicon produced in step d) is indicative of the presence of the Brunhilde poliovirus strain.

In a further embodiment of the method according to the invention, the method comprises the further the step of selective hybridisation of an oligonucleotide to a polioviral nucleic acid in the sample, whereby the oligonucleotide is selective for one or more poliovirus strains selected from the group consisting of: the MEF-1 type 2 strain or the Lansing strain, the Sabin type 2 strain, the Saukett H or G strains, and the Sabin type 3 or the Leon strains. Preferably in this embodiment the method comprises the steps of: a) amplifying at least a portion of polioviral nucleic acid in the sample with a primer pair that is specific for one or more poliovirus strains selected from the group consisting of: the MEF-1 type 2 strain or the Lansing strain, the Sabin type 2 strain, the Saukett H or G strains, and the Sabin type 3 or the Leon strains; and, b) detecting whether an amplicon is obtained in step a), whereby an amplicon produced with the primer pair specific for one or more of the poliovirus strains is indicative for the presence of those poliovirus strains. Preferably, in step a) of this method the portion of polioviral nucleic acid is amplified with at least one primer pair selected from the group consisting of: I) a forward primer comprising at least 12 contiguous nucleotides and the 3′-end of the sequence GGTTGTTGAGGGAGTCACGAGA and a reverse primer comprising at least 12 contiguous nucleotides and the 3′-end of the sequence CCCTGTCTCTACGGCTGTTAGC; II) a forward primer comprising at least 12 contiguous nucleotides and the 3′-end of the sequence GCAATTACGCCGCAAGC and a reverse primer comprising at least 12 contiguous nucleotides and the 3′-end of the sequence GTGTAGGTGCTCCTGGAGGT; III) a forward primer comprising at least 12 contiguous nucleotides and the 3′-end of the sequence AAGGAATTGGTGACATGATTGAGG and a reverse primer comprising at least 12 contiguous nucleotides and the 3′-end of the sequence CTCGGCTTTGTGTCAGGC; and, IV) a forward primer comprising at least 12 contiguous nucleotides and the 3′-end of the sequence AATGACCAGATTGGTGATTCCTTG and a reverse primer comprising at least 12 contiguous nucleotides and the 3′-end of the sequence GTAAATGCGGACTTTGGAGGTTACT; and whereby in step b) an amplicon produced with the primer pair in I) is indicative of the presence of the MEF-1 type 2 strain or the Lansing strain; an amplicon produced with the primer pair in II) is indicative of the presence of the Sabin type 2 strain; an amplicon produced with the primer pair in III) is indicative of the presence of the Saukett H or G strains; and an amplicon produced with the primer pair in IV) is indicative of the presence of the Sabin type 3 or the Leon strains.

In the methods according to the invention, an amplicon is, preferably detected by hybridisation with a fluorescent or chemiluminescent probe comprising a sequence that is complementary to a sequence in the amplicon, and whereby, preferably the detection is in real time.

Also, in the methods according to the invention, wherein prior to the selective hybridisation, ligation and/or amplification step(s), the method can further comprises the steps of purifying RNA of the poliovirus in the sample and reverse transcribing the polioviral RNA to provide a polioviral cDNA.

In a second aspect, the invention pertains to an oligonucleotide, primer or probe as defined herein for use in any of the above methods of the invention.

In a second aspect, the invention pertains to a kit comprising at least one of the above defined oligonucleotides, primers and probes, and optionally further comprising at least one of an enzyme, a solution, a buffer and an instruction manual for carrying out the methods of the invention.

### DESCRIPTION OF THE INVENTION

The present invention relates to methods for identifying and/or distinguishing polioviral strains, in particular polioviral strains used in vaccine production. The methods are based on selective hybridisation with oligonucleotides, i.e. primers and/or probes, that allow to distinguish between closely related but different polioviral strains on the basis of nucleotide polymorphisms existing between those polioviral strains.

Poliovirus are enteroviruses that infect humans. Three serotypes of poliovirus are known, serotypes 1, 2 and 3 or PV1, PV2, and PV3. For each serotype both virulent and attenuated poliovirus strains are available for vaccine production (see e.g. Table 1). Although for most polioviral strain is it is possible to design specific oligonucleotide primers or probes that allow to distinguish these strains from other polioviral strains, for two important serotype 1 polioviral vaccine strains, i.e. the virulent Mahoney strain and the attenuated Sabin type 1 strain, this has proven difficult because of the high level of nucleotide sequences identity between the viral genomes of these two strains. The inventors have focussed on 2 double nucleotide polymorphisms existing between the genomes of these polioviral strains for design of primers that allow to distinguish between the Mahoney and the Sabin type 1 strains. These double nucleotide polymorphism are present at positions 1942-1944 and 3894-3896 in SEQ ID NO: 1, the genomic sequence of the Mahoney poliovirus. The first double polymorphism at positions 1942 and 1944 in SEQ ID NO: 1 is unique for the Sabin type 1 strain; the latter double polymorphism at positions 3894 and 3896 in SEQ ID NO: 1 is unique for the Mahoney strain.

Thus, in a first aspect the invention relates to a the identification of a poliovirus strain in a sample, wherein the method allows to distinguish between a polio virus strain selected from the group consisting of Mahoney, Brunhilde, CHAT and Cox on the one hand, and, the Sabin type 1 poliovirus strain on the other hand.

The method preferably comprises a step of selective hybridisation of an oligonucleotide to a polioviral nucleic acid in the sample. The selective hybridisation of the oligonucleotide to the polioviral nucleic acid is understood to mean that the oligonucleotide forms a productive or positive duplex with the target polioviral nucleic acid, i.e. the polioviral nucleic acid with which the oligonucleotide has most complementarity, and not with the nucleic acid of other polio viruses that have less complementarity with the oligonucleotide. Selective hybridisation is thus performed under hybridisation conditions which promote the formation of a productive or positive duplex of the oligonucleotide with the target polioviral nucleic acid, while under these hybridisation conditions no productive or positive duplex is formed between the oligonucleotide and the non-target polioviral nucleic acids.

In the method of the invention the selective hybridisation of the oligonucleotide comprising the first double polymorphism is used to distinguish between a polio virus strain selected from the group consisting of Mahoney, Brunhilde, CHAT and Cox on the one hand, and, the Sabin type 1 poliovirus strain on the other hand. Thus, an oligonucleotide of the invention comprising the double polymorphism 1942 and 1944 in SEQ ID NO: 1 that selectively hybridises to a nucleic acid of the Mahoney poliovirus strain will not productively or positively hybridise to a nucleic acid of the Sabin type 1 poliovirus strain. Vice versa, an oligonucleotide of the invention comprising the double polymorphism 1942 and 1944 in SEQ ID NO: 1 that selectively hybridises to a nucleic acid of the Sabin type 1 poliovirus strain will not productively or positively hybridise to a nucleic acid of the Mahoney, Brunhilde, CHAT and Cox poliovirus strains.

In the method of the invention the selective hybridisation of the oligonucleotide comprising the double polymorphism at positions 3894 and 3896 in SEQ ID NO: 1 is used to distinguish between the Mahoney polio virus strain on the one hand, and, a poliovirus strain selected from the group consisting of Sabin type 1, Brunhilde, CHAT and Cox, on the other hand. Thus, an oligonucleotide of the invention comprising the double polymorphism at positions 3894 and 3896 in SEQ ID NO: 1 that selectively hybridises to a nucleic acid of the Mahoney poliovirus strain will not productively or positively hybridise to a nucleic acid of the Sabin type 1, Brunhilde, CHAT and Cox poliovirus strains. Vice versa, an oligonucleotide of the invention comprising the double polymorphism at positions 3894 and 3896 in SEQ ID NO: 1 that selectively hybridises to a nucleic acid of the Sabin type 1 poliovirus strain will not productively or positively hybridise to a nucleic acid of the Mahoney poliovirus strain.

The formation of a productive or positive duplex, i.e. the productive or positive hybridisation of an oligonucleotide of the invention to a polioviral nucleic acid is understood as the formation of a duplex between the oligonucleotide and the target polioviral nucleic acid that can be detected by the formation of an amplicon in an amplification or ligation-amplification assay (see below). In practice this will mean that the end of the oligonucleotide comprising the double polymorphism will form a duplex with the target polioviral nucleic acid, such that the oligonucleotide can be elongated by a polymerase or ligated to an adjacently base paired poly- or oligonucleotide molecule. As used herein, an ‘amplicon’ relates to a double stranded nucleic acid segment having a defined size and sequence that results from an amplification procedure, such as a PCR procedure. The size of the amplicon is governed by the sites on the two strands of a nucleic acid duplex to which the primers bind. As explained in U.S. Pat. No. 4,683,195, that segment of the product nucleic acid becomes the prevalent product of the amplification procedure after a small number of cycles of amplification.

As used herein the terms ‘specific to’ or ‘selective for’ a target sequence, in relation to a nucleic acid sequence such as an oligonucleotide sequence, relate to a nucleotide sequence that hybridises, under conditions used in given experimental circumstances, to the target nucleic acid but does not hybridize under those circumstances to sequences that are not target sequences. Nucleotide sequences that are specific for a particular polioviral target sequence are those that include bases all of which are complementary to the corresponding base on the target. Further as used herein, ‘specificity’ of a nucleic acid sequence for a target sequence also encompasses nucleic acids and oligonucleotides having a small number of nucleotides which may not be complementary to the corresponding nucleotides of the target sequence. Such sequences are still ‘specific’ or ‘selective’ for the target sequence, as used herein, as long as the extent of deviation from complementarity remains functionally of no consequence. In particular, such a sequence is ‘specific’ or ‘selective’ for the target sequence as long as it hybridises effectively to the target sequence but does not hybridise to any sequence that is not a target sequence, under the conditions used in given experimental circumstances.

The term ‘polioviral nucleic acid’ is herein understood to refer to a polioviral RNA or any part or fragment thereof, cDNA copies thereof, including their double stranded forms as well as either one of the single strands thereof.

The term ‘complement’ or ‘complementary sequence’ of a first sequence is herein understood to mean the second sequence that can form a double-stranded structure or duplex with the first sequence by matching base pairs, e.g. the complementary sequence to G-T-A-C is C-A-T-G.

In the method of the invention, the oligonucleotide that is used for selective hybridisation preferably is at least one of:

a) an oligonucleotide comprising at least 12 contiguous nucleotides of SEQ ID NO: 1 (=Mahoney sequence) or its complement and wherein the oligonucleotide comprises the sequence of positions 1942-1944 of SEQ ID NO: 1 or its complement at the 5′ or 3′ end;

b) an oligonucleotide comprising at least 12 contiguous nucleotides of SEQ ID NO: 1 or its complement and wherein the oligonucleotide comprises the sequence of positions 3894-3896 of SEQ ID NO: 1 or its complement at the 5′ or 3′ end;

c) an oligonucleotide comprising at least 12 contiguous nucleotides of SEQ ID NO: 2 (=Sabin type 1 sequence) or its complement and wherein the oligonucleotide comprises the sequence of positions 1942-1944 of SEQ ID NO: 2 or its complement at the 5′ or 3′ end; and,

d) an oligonucleotide comprising at least 12 contiguous nucleotides of SEQ ID NO: 2 or its complement and wherein the oligonucleotide comprises the sequence of positions 3894-3896 of SEQ ID NO: 2 or its complement at the 5′ or 3′ end.

In the method of the invention preferably at least one of a Mahoney-specific oligonucleotide as defined in a) and b), and a Sabin type 1-specific oligonucleotide as defined in c) and d) is used for selective hybridisation. In a preferred embodiment more than one or all four of the oligonucleotides as defined in a), b), c) and d) is used for selective hybridisation.

In the method of the invention, selective hybridisation with the oligonucleotide in a) is indicative of the presence of a poliovirus strain selected from the group consisting of Mahoney, Brunhilde, CHAT and Cox; selective hybridisation with the oligonucleotide in b) is indicative of the presence of the Mahoney poliovirus strain; selective hybridisation with the oligonucleotide in c) is indicative of the presence of the Sabin type 1 poliovirus strain; and, selective hybridisation with the oligonucleotide in d) is indicative of the presence of a poliovirus strain selected from the group consisting of Sabin type 1, CHAT and Cox. Thus, the poliovirus serotype 1 vaccine strains can be identified according to the scheme in Table A.

TABLE A

| | |
|---|---|
| | Oligonucleotide |
| | producing |
| | selective |
| poliovirus | hybridisation |

| type 1 strain | a) | b) | c) | d) |
|---|---|---|---|---|
| Mahoney | + | + | | |
| Sabin | | | + | + |
| Brunhilde | + | | | |
| CHAT | + | | | + |
| Cox | + | | | + |

The length of the contiguous sequence of SEQ ID NO: 1 or 2 in the oligonucleotides used for selective hybridisation preferably at least 12 contiguous nucleotides of SEQ ID NO: 1 or 2. More preferably, the oligonucleotides comprise at least 13, 14, 15, 16, 17, 18, 19, 20, 21, 22, 23 or 24 contiguous nucleotides of SEQ ID NO: 1 or 2. However, preferably the length of the contiguous sequence of SEQ ID NO: 1 or 2 in the oligonucleotides is not more than 25, 30, 40 contiguous nucleotides of SEQ ID NO: 1 or 2. The overall length of the oligonucleotides does not need be more than 60, 50, 40, 30 or 26 nucleotides.

In a preferred embodiment of the method of the invention, the oligonucleotide used for selective hybridisation comprises a mismatch in the contiguous sequence of SEQ ID NO: 1 or 2, whereby the mismatch preferably is a mismatch to both SEQ ID NO: 1 and 2, or their complements. The mismatch is introduced to destabilise the duplex between the oligonucleotide and the polioviral nucleic acid. The destabilising effect of the mismatch will be greater on the duplex between the oligonucleotide and the non-target polioviral nucleic acids compared to its effect on the duplex between the oligonucleotide and the target polioviral nucleic acid. The mismatch thereby enhances the selectivity of the hybridisation of the oligonucleotide to its target polioviral nucleic acid. Preferably the mismatch is present at a position in the contiguous sequence that is no more than 5, 4, 3 or 2 from the sequence of positions 1942-1944 or 3894-3896 of SEQ ID NO: 1, or their complements. More preferably, the mismatch in the contiguous sequence is at positions 1940, 1946, 3892 or 3898 of SEQ ID NO: 1 or its complement. It is understood herein that while the mismatch formally disrupts the sequence contiguous to SEQ ID NO: 1 or 2 in the oligonucleotide, the mismatch is ignored for the purpose of defining the contiguous sequence and its length in the oligonucleotides of the invention.

In a further embodiment of the invention, an oligonucleotide is used for selective hybridisation that allows to distinguish the Brunhilde strain from the other type 1 polioviral strains. This oligonucleotide is as the oligonucleotides for selective hybridisation described above except that in this oligonucleotide, the nucleotides corresponding to positions 3893 and 3896 of SEQ ID NO: 1 differ from both SEQ ID NO's: 1 and 2, or their complements. At the position corresponding to position 3893 of SEQ ID NO: 1, the Brunhilde-specific oligonucleotide comprises an A, or a T or U in the complementary Brunhilde-specific oligonucleotide and at position corresponding to position 3896 of SEQ ID NO: 1, the Brunhilde-specific oligonucleotide comprises a G, or a C in the complementary Brunhilde-specific oligonucleotide.

In the methods of the invention, the selective hybridisation of the oligonucleotide is preferably detected by an amplification or an amplification-ligation assay. Thus the selectively hybridising oligonucleotide may be one of the two primers in a primer pair for a nucleic acid amplification reaction such as PCR. Alternatively, the selectively hybridising oligonucleotide may be first ligated to one or more further oligonucleotides that are at least partially complementary to the target polioviral nucleic acid, after which the ligation product is amplified. Nucleic acid amplification methods usually employ two primers, dNTP's, and a (DNA) polymerase. A preferred method for amplification is PCR. PCR protocols are well known in the art, and are described in standard laboratory textbooks, “Molecular Cloning: A Laboratory Manual,” 2nd ed., Sambrook, Fritsch and Maniatis, Cold Spring Harbor Laboratory, Cold Spring Harbor, N.Y., 1989; “Current Protocols in Molecular Biology,” Ausubel et al., John Wiley and Sons, New York 1987 (updated quarterly); and “PCR Protocols: A Guide to Methods and Applications,” Innis et al., Academic Press, San Diego, Calif. 1990. Other multiplex and/or isothermal amplification methods that may be applied include e.g. LCR, self-sustained sequence replication (3SR), Q-β-replicase mediated RNA amplification, rolling circle amplification (RCA) or strand displacement amplification (SDA). In some instances this may require replacing the primer-binding sites in the tags of the probes by a suitable (RNA) polymerase-binding site.

Amplification-ligation assays such as multiplex ligation amplification, ligase detection reactions (LDR) or multiplex ligation-dependent probe amplification (MLPA) are e.g. described in WO 96/15271, WO 97/45559, and WO 01/61033. In ligation-amplification assays the respective 5′- and 3′-ends of a pair of a first and second oligonucleotide probes that are annealed essentially adjacent to the complementary parts of a polioviral target sequence are connected to form a covalent bond by any suitable means known in the art. The ends of the probes are preferably connected enzymatically in a phosphodiester bond by a ligase, preferably a DNA ligase. DNA ligases are enzymes capable of catalysing the formation of a phosphodiester bond between (the ends of) two polynucleotide strands bound at adjacent sites on a complementary strand. DNA ligases usually require ATP (EC 6.5.1.1) or NAD (EC 6.5.1.2) as a cofactor to seal nicks in double stranded DNA. Suitable DNA ligase for use in the present invention are T4 DNA ligase, E. coli DNA ligase or preferably a thermostable ligase like e.g. Thermus aquaticus (Taq) ligase, Thermus thermophilus DNA ligase, or Pyrococcus DNA ligase. Alternatively, chemical autoligation of modified polynucleotide ends may be used to ligate two oligonucleotide probes annealed at adjacent sites on the complementary parts of a target sequence (Xu and Kool, 1999, Nucleic Acid Res. 27: 875-881).

The sample in the method of the invention, may be any sample suspected to contain a polioviral strain. The sample can be a sample obtained from any stage of a poliovirus vaccine production process, including e.g. samples from batches used for inoculation, or samples taken at various stages during culture, purification, inactivation and formulation of the vaccine. Alternatively, the sample may be a clinical sample or a clinical isolate obtained from a subject suspected of or suffering from a disease or syndrome that is at least partially caused by a poliovirus. The subject may also be an asymptomatic individual considered to be at risk of a polioviral infection. The sample may be a cellular sample such as a tissue sample, e.g., a sample of lung tissue obtained as a biopsy or post-mortem, a fluid sample such as blood, saliva, sputum, urine, cerebrospinal fluid, or a swabbed sample obtained by swabbing a mucus membrane surface such as a nasal surface, a pharyngeal surface, a buccal surface, and the like, or it may be obtained from an excretion such as feces, or it may be obtained from other bodily tissues or body fluids commonly used in clinical diagnostic testing. The sample can be obtained from a human subject or from a non-human mammalian subject. In a preferred embodiment of the method, the sample is suspected to contain a polioviral strain selected form the group consisting of Mahoney, Sabin type 1, Brunhilde, CHAT and Cox.

In a preferred embodiment, the method of the invention comprises the step of: a) amplifying at least a portion of polioviral nucleic acid in the sample with a primer pair comprising a forward primer that is at least one of:
(i) a forward primer comprising at least 12 contiguous nucleotides and the 3′-end of the sequence: 5′-CCCTTTGACTTAAGTHCCAC-3′ (SEQ ID NO: 3), wherein H is a nucleotide that is incapable of base pairing with C;
(ii) a forward primer comprising at least 12 contiguous nucleotides and the 3′-end of the sequence: 5′-CCCTTTGACTTAAGTHCAAA-3′ (SEQ ID NO: 4), wherein H is a nucleotide that is incapable of base pairing with C;
(iii) a forward primer comprising at least 12 contiguous nucleotides and the 3′-end of the sequence: 5′-CCATGGTGTTCTTTTVTGTG-3′ (SEQ ID NO: 5), wherein V is a nucleotide that is incapable of base pairing with A;
(iv) a forward primer comprising at least 12 contiguous nucleotides and the 3′-end of the sequence: 5′-CCATGGTGTTCTTTTVTTTT-3′ (SEQ ID NO: 6), wherein V is a nucleotide that is incapable of base pairing with A;
(v) a forward primer comprising at least 12 contiguous nucleotides and the 3′-end of the sequence: 5′-GATTTACTCAGCAGAVTAGC-3′ (SEQ ID NO: 7), wherein V is a nucleotide that is incapable of base pairing with A;
(vi) a forward primer comprising at least 12 contiguous nucleotides and the 3′-end of the sequence: 5′-GATTTACTCAGCAGAVTGGA-3′ (SEQ ID NO: 8), wherein V is a nucleotide that is incapable of base pairing with A;
(vii) a forward primer comprising at least 12 contiguous nucleotides and the 3′-end of the sequence: 5′-AACTCTGTTATTTTGVCGCT-3′ (SEQ ID NO: 9), wherein V is a nucleotide that is incapable of base pairing with A; and,
(viii) a forward primer comprising at least 12 contiguous nucleotides and the 3′-end of the sequence: 5′-AACTCTGTTATTTTGVCTCC-3′ (SEQ ID NO: 10), wherein V is a nucleotide that is incapable of base pairing with A;
   and a reverse primer, whereby a reverse primer in a pair with a forward primer produces an amplicon with the forward primers (i), (iii), (v) and (vii) on a reference cDNA template comprising the sequence of a Mahoney poliovirus strain or with the forward primer (ii), (iv), (vi) and (viii) on a reference cDNA template comprising the sequence of a Sabin type 1 poliovirus strain.

In this preferred embodiment, the method of the invention comprises the further step of: b) detecting whether an amplicon is obtained in step a).

In step b): an amplicon produced with at least one of forward primers (i) and (iii) is indicative of the presence of a poliovirus strain selected from the group consisting of Mahoney, Brunhilde, CHAT and Cox; an amplicon produced with at least one of forward primers (ii) and (iv) is indicative of the presence of the Sabin type 1 poliovirus strain; an amplicon produced with at least one of forward primers (v) and (vii) is indicative of the presence of the Mahoney poliovirus strain; and, an amplicon produced with at least one of forward primers (vi) and (viii) is indicative of the presence of a poliovirus strain selected from the group consisting of Sabin type 1, CHAT and Cox. Thus, the poliovirus serotype 1 vaccine strains can be identified according to the scheme in Table B.

TABLE B

| | |
|---|---|
| | Oligonucleotide producing |
| poliovirus | selective hybridisation |

| type 1 strain | i and iii | v and vii | ii and iv | vi and viii |
|---|---|---|---|---|
| Mahoney | + | + | | |
| Sabin | | | + | + |
| Brunhilde | + | | | |
| CHAT | + | | | + |
| Cox | + | | | + |

This embodiment of the method of the invention may as an alternative or in addition comprise a step d) of amplifying at least a portion of polioviral nucleic acid in the sample with a primer pair comprising a forward primer that is at least one of:
(ix) a forward primer comprising at least 12 contiguous nucleotides and the 3′-end of the sequence: 5′-GATTTACTCAGCAGATAGGG-3′ (SEQ ID NO: 32);
(x) a forward primer comprising at least 12 contiguous nucleotides and the 3′-end of the sequence: 5′-AACTCTGTTATTTTGVCCCC-3′ (SEQ ID NO: 33), wherein V is a nucleotide that is incapable of base pairing with A;

and a reverse primer, whereby a reverse primer in a pair with a forward primer produces an amplicon with the forward primers (ix) and (x) on a reference cDNA template comprising the sequence of the Brunhilde poliovirus strain; and a step e), of detecting whether an amplicon is obtained in step d), whereby an amplicon produced in step d) is indicative of the presence of the Brunhilde poliovirus strain.

In this embodiment of the method of the invention, at least a portion of polioviral nucleic acid in the sample amplified using a primer pair. The primer pair for amplification will usually comprise a forward primer and a reverse primer. As used herein the term ‘primer’ or ‘oligonucleotide primer’ relates to an oligonucleotide having a specific or desired nucleotide sequence which is substantially complementary to a particular sequence in the polioviral target sequence to be amplified. When the primer is caused to hybridise to the specific sequence in the target nucleic acid to which it is complementary, it may serve as the priming position, or the initiation position, for the action of a primer-dependent DNA polymerase. The primer, once hybridised, acts to define the 5′ end of the operation of the elongation activity of the polymerase on the template nucleic acid. Commonly in PCR, a specific pair of primers is employed, wherein one of the primers hybridises to the target nucleic acid or to one of the strands thereof and the second primer hybridises to the strand elongated from the first primer and/or to the complementary strand in the target nucleic acid. The primers hybridise in such an orientation that elongation by the polymerase, which proceeds in the direction from 5′- to 3′-, is in the direction leading from each primer toward the site of hybridisation of the other primer. After several rounds of hybridisation and elongation a segment of DNA is exponentially amplified, having a defined length whose ends are defined by the sites to which the primers hybridise. In the context of the invention, if one of the primers in a primer pair for amplification is referred to as a ‘forward primer’, the other primer in the pair is the ‘reverse primer’ that is complementary to a DNA sequence elongated from the forward primer on the target nucleic acid as template for the polymerase.

In this embodiment of the method of the invention, the forward primers comprise at least 12 contiguous nucleotides and the 3′-end of the sequences defined in i)-viii) above. Preferably however, the forward primers comprise at least 13, 14, 15, 16, 17, 18, 19 or 20 contiguous nucleotides and the 3′-end of the sequences defined in i)-viii). In the forward primers, H is not G and V is not or U. H is thus preferably A, C, T or U or any other nucleotide or analogue thereof that is incapable of base pairing with C. Likewise, V is preferably A, G or C or any other nucleotide or analogue thereof that is incapable of base pairing with A.

In this embodiment of the method of the invention, the reverse primer preferably comprises at its 3′-end a sequence of at least 12, 13, 14, 15, 16, 17, 18, 19 or 20 contiguous nucleotides that are complementary to a sequence in an elongation product obtained on a polioviral template with a forward primer defined above. More preferably, the contiguous nucleotides complementary to the sequence in the elongation product obtained on a polioviral template with a forward primer are less than 1000, 750, 500 or 400 nucleotides from the contiguous sequence of the forward primer in the polioviral template. The complementary contiguous nucleotides in the reverse primer are preferably chosen in an area in the polioviral sequence where there are no or only 1, 2, 3 or 4 nucleotide polymorphisms between the Mahoney and Sabin type 1 sequences. More preferably, the complementary contiguous nucleotides in the reverse primer are chosen in an area in the polioviral sequence where there are no or only 1, 2, 3 or 4 nucleotide polymorphisms between any of the sequences of the Mahoney, Sabin type 1, Brunhilde, CHAT and Cox polioviral strains. The reference cDNA template may be any cDNA comprising a fragment of the sequence of a Mahoney or Sabin type 1 poliovirus genomes that includes the sequences of both the forward and reverse primers in question.

In a further preferred embodiment of the method of the invention, the forward primer that is at least one of the forward primers defined in (i) and (ii) above, and the reverse primer is 5′-GATCCTGCCCAGTGTGTGTAG-3′ (SEQ ID NO: 11).

In a further embodiment of the above methods of the invention, the method further comprises a step of selective hybridisation of an oligonucleotide to a polioviral nucleic acid in the sample, the oligonucleotide is specific or selective for one or more poliovirus strains selected from the group consisting of: the MEF-1 type 2 strain or the Lansing strain, the Sabin type 2 strain, the Saukett H or G strains, and the Sabin type 3 or the Leon strains. The method may further be a method as described above for the serotype 1 polioviral strains. Preferably, the method comprises the steps of: a) amplifying at least a portion of polioviral nucleic acid in the sample with a primer pair that is specific for one or more poliovirus strains selected from the group consisting of: the MEF-1 type 2 strain or the Lansing strain, the Sabin type 2 strain, the Saukett H or G strains, and the Sabin type 3 or the Leon strains; and, b) detecting whether an amplicon is obtained in step a), whereby an amplicon produced with the primer pair specific for one or more of the poliovirus strains is indicative for the presence of those poliovirus strains. In a preferred embodiment of the method, in step a) the portion of polioviral nucleic acid is amplified with at least one primer pair selected from the group consisting of: I) a forward primer comprising at least 12 contiguous nucleotides and the 3′-end of the sequence GGTTGTTGAGGGAGTCACGAGA (SEQ ID NO: 12) and a reverse primer comprising at least 12 contiguous nucleotides and the 3′-end of the sequence CCCTGTCTCTACGGCTGTTAGC (SEQ ID NO: 13); II) a forward primer comprising at least 12 contiguous nucleotides and the 3′-end of the sequence GCAATTACGCCGCAAGC (SEQ ID NO: 14) and a reverse primer comprising at least 12 contiguous nucleotides and the 3′-end of the sequence GTGTAGGTGCTCCTGGAGGT (SEQ ID NO: 15); III) a forward primer comprising at least 12 contiguous nucleotides and the 3′-end of the sequence AAGGAATTGGTGACATGATTGAGG (SEQ ID NO: 16) and a reverse primer comprising at least 12 contiguous nucleotides and the 3′-end of the sequence CTCGGCTTTGTGTCAGGC (SEQ ID NO: 17); and, IV) a forward primer comprising at least 12 contiguous nucleotides and the 3′-end of the sequence AATGACCAGATTGGTGATTCCTTG (SEQ ID NO: 18) and a reverse primer comprising at least 12 contiguous nucleotides and the 3′-end of the sequence GTAAATGCGGACTTTGGAGGTTACT (SEQ ID NO: 19); and, whereby in step b) an amplicon produced with the primer pair in I) is indicative of the presence of the MEF-1 type 2 strain or the Lansing strain; an amplicon produced with the primer pair in II) is indicative of the presence of the Sabin type 2 strain; an amplicon produced with the primer pair in III) is indicative of the presence of the Saukett H or G strains; and an amplicon produced with the primer pair in IV) is indicative of the presence of the Sabin type 3 or the Leon strains. The forward and reverse primers comprise at least 12 contiguous nucleotides and the 3′-end of the sequences defined in I)-IV) above. Preferably however, the forward primers comprise at least 13, 14, 15, 16, 17, 18, 19 or 20 contiguous nucleotides and the 3′-end of the sequences defined in I)-IV) above.

In the above methods of the invention, the amplicon is detected by means and method well known in the art per se, including e.g. the use of fluorescent or chemiluminescent labels. Preferably, an amplicon is detected by hybridisation with a fluorescent or chemiluminescent probe comprising a sequence that is complementary to a sequence in the amplicon. More preferably, the amplicon is detection in real time. Preferred probes for detection of amplicons obtained with preferred forward and reverse primers of the invention include probes comprising at least 13, 14, 15, 16, 17, 18, 19 or 20 contiguous nucleotides of a sequence that is complementary to any one of SEQ ID NO: 26 (Sabin 1 amplicon), SEQ ID NO: 27 (Mahoney amplicon), SEQ ID NO: 28 (Sabin 2 amplicon), SEQ ID NO: 29 (MEF-1 amplicon), SEQ ID NO: 30 (Sabin 3 amplicon) and SEQ ID NO: 31 (Saukett H amplicon), or their complements, including e.g. the probes of SEQ ID NO's: 20-24. Suitable label fluorescent labels are e.g. listed at www.isogen-lifescience.com. Preferred fluorescent labels include the labels and quencher used in the Examples herein.

In the above methods of the invention, preferably, prior to the steps of selective hybridisation, amplification and/or ligation, the method further comprises the steps of purifying RNA of the poliovirus in the sample and reverse transcribing the polioviral RNA to provide a polioviral cDNA.

Purification of RNA as a step in the methods of the invention, in particular, as a step leading up to a RT-PCR procedure, relates to releasing RNA from a latent or inaccessible form in a virion or a cell and allowing the RNA to become freely available. In such a state, it is suitable for effective amplification by reverse transcription and use of the amplification and, where appropriate, ligation reactions. Releasing RNA may include steps that achieve the disruption of virions containing viral RNA, as well as disruption of cells that may harbour such virions. Purification of RNA is generally carried out under conditions that rigorously and effectively exclude or inhibit any ribonuclease activity that may be present. Additionally, purification of RNA may include steps that achieve at least a partial separation of the RNA dissolved in an aqueous medium from other cellular or viral components, wherein such components may be either particulate or dissolved.

In the methods of the invention, ‘reverse transcription’ or ‘RT’ relates to a procedure catalyzed by an enzyme activity, reverse transcriptase, that synthesizes a cDNA from a single stranded RNA molecule, with the use of oligonucleotide primers having free 3′-hydroxyl groups. The oligonucleotide primers may either have specific sequences complementary to the polioviral target RNA, such as e.g. the forward and reverse primers exemplified herein above. Alternatively, the RT reaction may be primed using short random oligonucleotide primers.

Another aspect of the invention relates to the oligonucleotides, primers and probes defined herein above for use in the methods of the invention. In particular in this aspect the invention relates to sets of forward and reverse primers and optionally a labelled probe for detection of the amplicon obtained with the forward and reverse primers.

The present invention also finds embodiments in the form of kits. Kits according to the invention include e.g. a kit of parts consisting of one or more containers comprising oligonucleotides, primers and probes suitable for use in the methods of the invention. The kits can further comprise (containers comprising) enzymes, solutions, buffers and a manual with instructions, for carrying out the methods of the invention.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

All patent and literature references cited in the present specification are hereby incorporated by reference in their entirety.

The following examples are offered for illustrative purposes only, and are not intended to limit the scope of the present invention in any way.

### DESCRIPTION OF THE FIGURES

FIG. 1. Optimisation of the identification test for poliovirus type 1 strains. (A) Real-time PCR with specific forward primers for Sabin type 1 and Sabin type 1 strain as template; (B) specific primers for Sabin type 1 and Mahoney strain as template; (C) specific primers for Mahoney and Mahoney strain as template; and (D) specific primers for Mahoney and Sabin type 1 strains as template. The specificity of the primers for exclusive identification of Sabin virus type 1 or Mahoney is improved by reducing the primer length.

FIG. 2. The identification test developed for six distinct poliovirus strains. Specific primer and probe sets were used in real-time PCR to identify a particular polio strain: Sabin type 1 (♦), Mahoney (▪), Sabin type 2 (▴), MEF-1(x), Sabin type 3 (*), Saukett H (), plasmids as positive control (+) and PCR grade water as a negative control (◯).

FIG. 3. The identification of Sabin type 1 polioviruses present in samples obtained from different stages of vaccine production: after harvest (♦), clarification (A), concentration (x), gel permeation chromatography (), ion-exchange chromatography (+) and formaldehyde inactivation (□). A plasmid with the amplicon sequence was used as a positive control (▪). Analyses with template obtained from Mahoney poliovirus (*) and without template (◯) were performed as negative controls.

### EXAMPLES

### Materials and Methods

Poliovirus samples were obtained from different stages of IPV production. IPV was produced with either wild-type strains or attenuated strains as described previously (van Wezel et al., 1978; van Wezel et al., 1979). Briefly, Vero cells were cultivated in fermenters on micro carriers. Vero cells were infected with wild-type polio strains: Mahoney (type 1), MEF-1 (type 2) or Saukett H (type 3), or with attenuated strains: Sabin type 1 (LSc 2ab KP2), Sabin type 2 (P712 Ch2ab-KP2) or Sabin type 3 (Pfizer 457-III) strains. After three or four days of cultivation, poliovirus was purified from the culture supernatant by clarification, gel permeation chromatography and ion-exchange chromatography. Prior to inactivation the fluid was filtered to remove large virus aggregates and formaldehyde was added for inactivation. Samples taken prior to formaldehyde inactivation were heated for 30′ at 60° C. to inactivate the polioviruses. Viral RNA was extracted and purified from 200 μl of these samples using a fully automated procedure (MagNA Pure Compact System, Roche). Extracted RNA samples were stored at −20° C. prior to analysis. Preparation and amplification of cDNA were performed in a real-time PCR apparatus (LightCycler 2.0; Roche).

A reaction mixture was prepared by using LightCycler RNA Master HybProbe kit (Roche). The mixture contained 7.5 μl RNA master, 3.2 μl H₂O, 1.3 μl of a 50 mM Mn(OAc)₂ solution, 2 μl probe, 2 μl of 5.0 μM forward primer, 2 μl of 5.0 μM reverse primer and 2 μl isolated RNA, plasmid (positive control) or PCR grade water (negative control). The final concentration of probe for MEF-1 was 0.4 μM, and the concentration of the remaining probes was 0.2 μM. The sequences of primers and probes used are listed in Table 3. Design of experiments was used as an approach to optimise the reaction conditions, such as annealing temperature, annealing time, ramp rate and reagent concentrations. Software for Design of Experiments was used for modelling and visualisation of research data and for the calculation of optimal conditions (MODDE 8.0.2., Umetrics; Sweden).

For a rapid and accurate identification of the vaccine-specific polio strains, the reverse transcription step was executed in the PCR apparatus that was programmed to run for 20′ at 61° C. For polioviruses of type 1, the amplification step was performed for 30″ 95°; [5″ 95°; 1″ 55°; 7″ 72°]×35; 30″ 40° and ramp rate of 2° C./sec. For polioviruses of type 2, the amplification step was performed for 30″ 95°; [5″ 95°; 15″ 58°; 5″ 72°]×35; 30″ 40° and ramp rate of 20° C./sec. For polioviruses of type 3, the amplification step was performed for 30″ 95°; [5″ 95°; 15″ 56°; 5″ 72°]×35; 30″ 40° and ramp rate of 20° C./sec.

Six plasmids were prepared to be used as positive controls in the identification assays. The inserts were made by RT-PCR using Ready-to-Go RT-PCR beads (GE Healthcare Life Sciences). The reverse transcription was performed with a mixture of 11 μl viral RNA, 5 μl pd(N)₆ and 20 μl H₂O for 20′ at 42° C. and 5′ at 95° C. Then, 7 μl of a specific forward and a specific reverse primer was added to the mixture (Table 3). PCR was performed for 5′ 95°; [30″ 95°; 30″ 55°; 1′ 72°]×35; 7′ 72° and stored at 4° C. The PCR products were separated on a 1.5% agarose gel. The bands were excised and the DNA was purified using Gel Clean-Up kit (Promega). These inserts were cloned in a pGEM T easy vector system II (Promega) and transformed to E. coli Novablue cells (Novagen). Positive clones were selected and grown up overnight. in 10 ml LB medium (Sambrook, Fritsch et al. 1989). The plasmids were isolated and checked by PCR using the two specific primer combinations and gel electrophoresis. The selected plasmids were sequenced using a DNA sequencing kit (BigDye terminator cycle sequencing kit v3.1; Applied Biosystems) and DNA analyser (ABI Prism 310 DNA Analyzer; Applied Biosystems) and tested in real-time PCR to verify their suitability as positive controls.

### Results

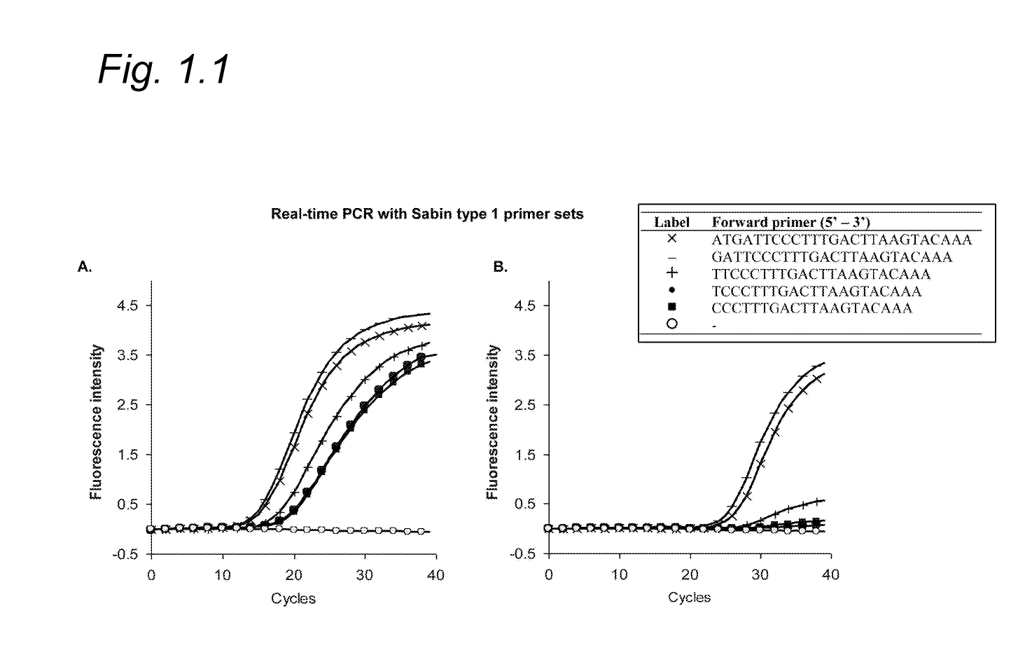

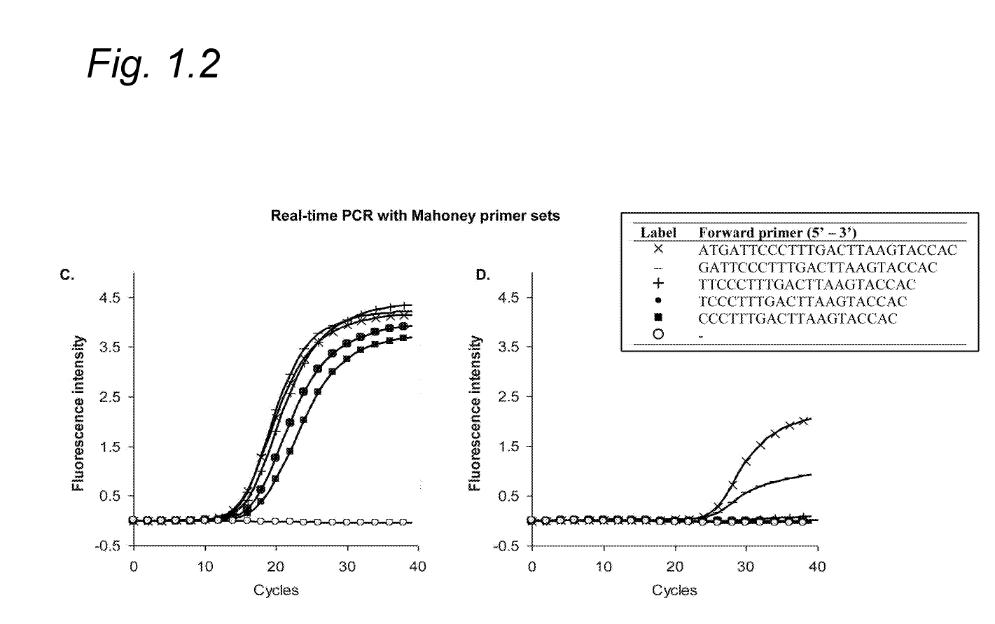

The poliovirus type 1 strains, Sabin and Mahoney, have a high degree of sequence homology (99.2%). Sabin type 1 contains only 58 single mutations if the sequence of 7441 nucleotides is compared to the Mahoney strain. Therefore, it is difficult to develop unique probe and primer sets that will selectively amplify cDNA of Sabin type 1 or Mahoney strain. Ten forward primers were designed either for Mahoney or for Sabin type 1 and tested for specificity (FIG. 1). The primers vary in length and have two point mutations close to the 3′ side. Furthermore an extra mismatch was incorporated in the forward primers to improve the specificity for the exclusive identification of Sabin type 1 or Mahoney strain. Results showed that the length of the primer is crucial for specific identification of these poliovirus strains (FIG. 1) and that it has to be 21 bases or less to prevent generation of an amplified product on both strains (see FIGS. 1B and 1D). Based on the results two forward primers were selected to be used for the identification of the Sabin type 1 or Mahoney strain (Table 3). The reverse primer and probe were shared for the identification of both poliovirus strains.

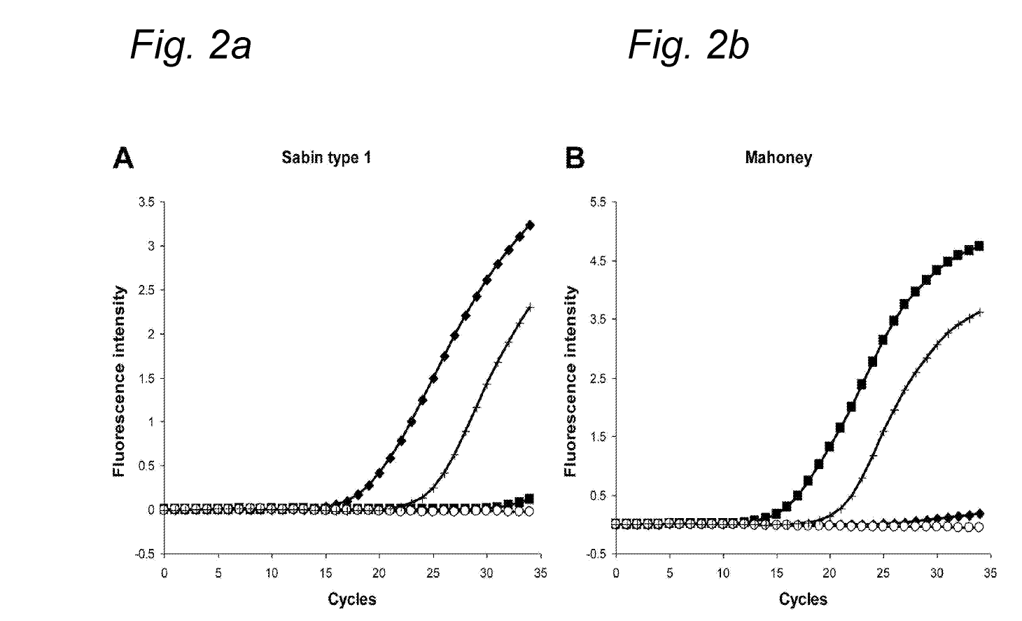

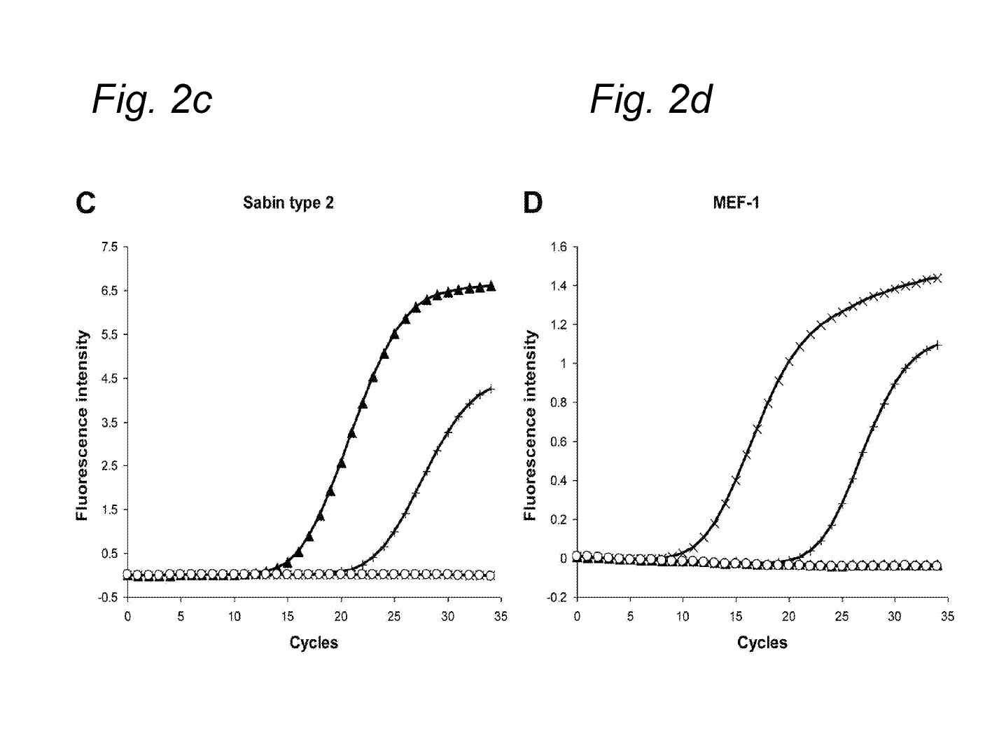

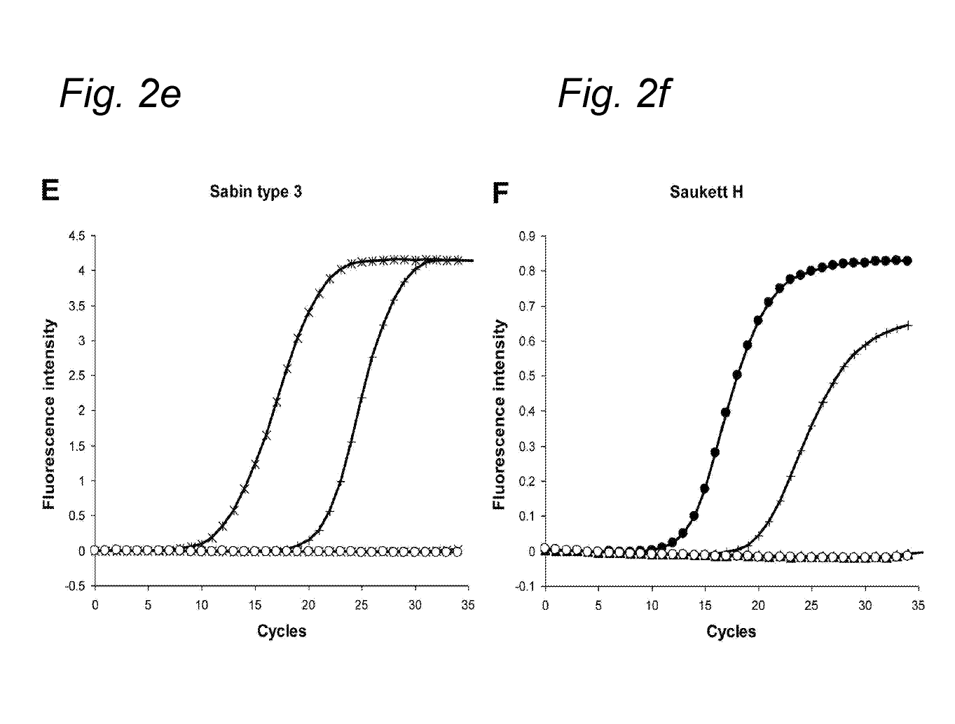

The sequence homology between Sabin type 2 and MEF-1 is only 82.5%, whereas the match between Sabin type 3 and Saukett H is 88.1%. As a consequence, development of unique probe and primer sets and was easier (Table 3). The real-time PCR showed that specific amplification occurred with these polio strains and their corresponding probe and primer sets. No cross-reactivity was observed when the probe and primer sets were applied to a different polio strain (FIG. 2).

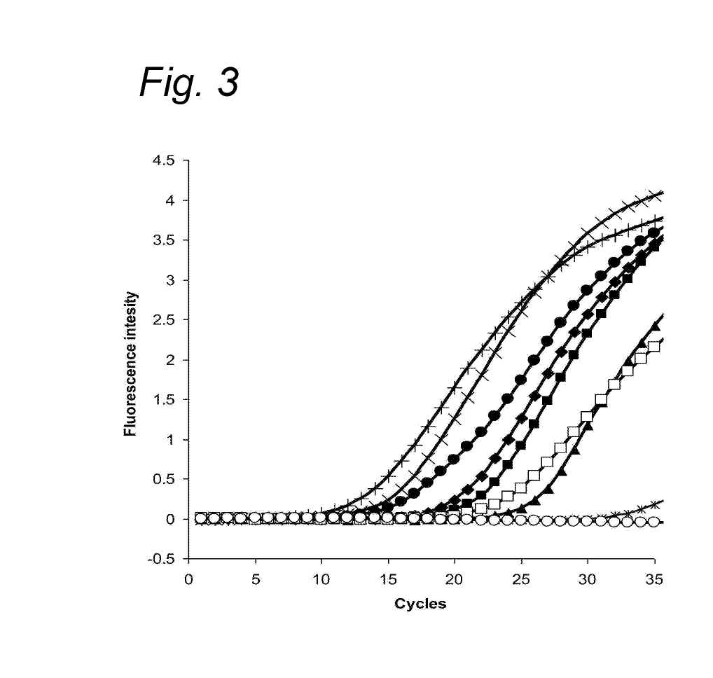

To assess its usability the identification test was performed on samples obtained from different stages in the production process, i.e. harvested, clarified, purified and formaldehyde-inactivated virus suspensions. The type of polioviruses could be identified in all stages of the production process, even on formaldehyde inactivated final bulk. An example is given in FIG. 3 of samples obtained during production of Sabin type 1. Similar results were obtained with the other vaccine-specific poliovirus strains.

Multiple whole genome and partial sequences of distinct poliovirus strains have been published in the literature. The sequences of primers and probes were compared with the literature data to reveal their specificity for homologous polioviruses. The homology search revealed that an amplicon will be formed with a limited number of poliovirus strains (Table 4). The alignment also showed that attenuated polioviruses type 1 strains Cox and CHAT, derived from Mahoney (Martin and Minor, 2002), probably will generate a positive signal in the real-time PCR if the primers and probe for the identification of Mahoney are used but not with the Sabin type 1 primers. Furthermore, the homology of 99.8% between the virulent MEF-1 and Lansing strains probably excludes unequivocal identification of the MEF-1 strain (La Monica, et al. 1986; Dragunsky et al., 2004). Presumably no distinction can be made between the virulent parent strain Leon/37 and the derived, attenuated Sabin type 3 strain (Leon 12 a1b) (Stanway et al., 1984).

The homology between Leon/37 and Sabin type 3 is 99.9%. A positive fluorescence signal is obtained with at least two circulating vaccine-derived polioviruses (VDPV). Positive results are also expected with many more circulating VDPV strains and the identification tests for Sabin strains. These VDVP strains are introduced in the field by immunisation with the OPV vaccine (Kew et al., 2005) and OPV is based on the Sabin strains. In that case discrimination between poliovirus strains with a high homology (>99%) can be performed only by partial or whole genome sequencing.

In conclusion, the PCR methods reported in the present study can be used for the accurate identification of poliovirus strains used for production of polio vaccines. The study revealed that the real-time PCR test discriminates between poliovirus strains, such as Mahoney, MEF-1, Saukett H, Sabin type 1, Sabin type 2 and Sabin type 3 and can be applied in different stages of vaccine production.

TABLE 1
Polioviruses used for vaccine production

| Virus | Serotype | Virulent |
|---|---|---|
| Mahoney | Type 1 | yes |
| MEF-1 | Type 2 | yes |
| Saukett H | Type 3 | yes |
| Sabin type 1 | Type 1 | no |
| Sabin type 2 | Type 2 | no |
| Sabin type 3 | Type 3 | no |

TABLE 2
Identification methods for polioviruses

| Method | Result | Advantage | Disadvantage |
|---|---|---|---|
| ELISA | Specific binding of | Easy to perform | Low specificity. Production of |
| | monoclonal or polyclonal | | specific antibodies is complex |
| | antiserum to poliovirus | | |
| Hybridisation | Sequence-specific | Accurate results | Test is laborious |
| | interaction between | | |
| | poliovirus RNA and a | | |
| | probe | | |
| PCR | Amplification of cDNA | Rapid and | Less detailed information |
| | generated from poliovirus | accurate results | than sequencing |
| Sequencing | Determination of the | Unambiguous | Complicated data analyses; |
| | (partial) sequence of the | identification of | test is laborious |
| | poliovirus | polio strain | |

TABLE 4
Homologous poliovirus strains¹⁾

| Mahoney | Sabin 1 | MEF-1 | Sabin 2 | Saukett H | Sabin 3 |
|---|---|---|---|---|---|
| Brunhilde | — | Lansing | — | Saukett G | P3/Leon 12 a₁b³⁾ |
| CHAT²⁾ | | | | | P3/Leon/37⁴⁾ |
| Cox²⁾ | | | | | |

¹⁾Polio strains with a high homology will probably generate a false-positive response in the identification test
²⁾Attenuated poliovirus derived from Mahoney
³⁾Different name for the Sabin type 3 strain
⁴⁾Virulent parent strain of Leon 12 a1b or Sabin type 3 strain

TABLE 3
Primer and probes

| Primer | | | | TM | |
|---|---|---|---|---|---|
| or probe | Sequence (5′-3′) | Orientation | Position | (° C.) | Specificity |
| MAHONEY-F | CCCTTTGACTTAAGTACCAC | forward | 1905-1924 | 55.3 | Mahoney |
| SABIN1-F | TCCCTTTGACTTAAGTACAAA | forward | 1904-1924 | 52.0 | Sabin type 1 |
| POLIO1-R | GATCCTGCCCAGTGTGTGTAG | reverse | 2083-2063 | 56.9 | Mahoney/Sabin type 1 |
| POLIO1-TM | FAM-AGGGTTCGGTTAAGTGACAAACCACATAC-BBQ¹⁾ | — | 1950-1978 | 63.4 | Mahoney/Sabin type 1 |
| MEF1-F | GGTTGTTGAGGGAGTCACGAGA | forward | 2505-2526 | 59.6 | MEF-1 |
| MEF1-R | CCCTGTCTCTACGGCTGTTAGC | reverse | 2631-2610 | 59.5 | MEF-1 |
| MEF1-TM | YAK-ACACCACTGACACCTGCCAACAACT-BBQ¹⁾ | — | 2536-2560 | 64.0 | MEF-1 |
| SAUKETT-F | GCAATTACGCCGCAAGC | forward | 2076-2092 | 57.8 | Saukett H |
| SAUKETT-R | GTGTAGGTGCTCCTGGAGGT | reverse | 2227-2208 | 56.6 | Saukett H |
| SAUKETT-TM | YAK-TTCGTGGTAACAGCCAACTTCACCA-BBQ | — | 2134-2158 | 65.0 | Saukett H |
| SABIN2-F | AAGGAATTGGTGACATGATTGAGG | forward | 2480-2503 | 58.7 | Sabin type 2 |
| SABIN2-R | CTCGGCTTTGTGTCAGGC | reverse | 2579-2562 | 57.4 | Sabin type 2 |
| SABIN2-TM | FAM-TGGAAGTCGGGGGAACCAATGC-BBQ | — | 2551-2530 | 67.1 | Sabin type 2 |
| SABIN3-F | AATGACCAGATTGGTGATTCCTTG | forward | 3134-3157 | 58.7 | Sabin type 3 |
| SABIN3-R | GTAAATGCGGACTTTGGAGGTTACT | reverse | 3253-3229 | 59.9 | Sabin type 3 |
| SABIN3-TM | FAM-TGTGATCATTGACAACACGAACTGCCAA-BBQ | — | 3218-3191 | 66.7 | Sabin type 3 |

¹⁾Abbreviations. FAM: Carboxyfluorescein, YAK: Yakima yellow, BBQ: BlackBerry Quencher

### REFERENCES

Boot, H. J., R. M. Schepp, et al. (2004). “Rapid RT-PCR amplification of full-length poliovirus genomes allows rapid discrimination between wild-type and recombinant vaccine-derived polioviruses.” J Virol Methods 116(1): 35-43.
Crawford, N. W. and J. P. Buttery (2010). “Poliomyelitis eradication: another step forward.” Lancet. 376(9753): 1624-1625.
Dragunsky, E. M., A. P. Ivanov, et al. (2004). “Evaluation of immunogenicity and protective properties of inactivated poliovirus vaccines: a new surrogate method for predicting vaccine efficacy.” J Infect Dis 190(8): 1404-1412.
Dutta, A. (2008). “Epidemiology of poliomyelitis—options and update.” Vaccine 26(45): 5767-5773.
Heymann, D. L., R. W. Sutter, et al. (2005). “A global call for new polio vaccines.” Nature 434(7034): 699-700.
Heymann, D. L., R. W. Sutter, et al. (2006). “A vision of a world without polio: the OPV cessation strategy.” Biologicals 34(2): 75-79.
Kew, O. M., R. W. Sutter, et al. (2005). “Vaccine-derived polioviruses and the endgame strategy for global polio eradication.” Annu Rev Microbiol 59: 587-635.
Kilpatrick, D. R., K. Ching, et al. (2004). “Multiplex PCR method for identifying recombinant vaccine-related polioviruses.” J Clin Microbiol 42(9): 4313-4315.
Kilpatrick, D. R., B. Nottay, et al. (1998). “Serotype-specific identification of polioviruses by PCR using primers containing mixed-base or deoxyinosine residues at positions of codon degeneracy.” J Clin Microbiol 36(2): 352-357.
Kilpatrick, D. R., B. Nottay, et al. (1996). “Group-specific identification of polioviruses by PCR using primers containing mixed-base or deoxyinosine residue at positions of codon degeneracy.” J Clin Microbiol 34(12): 2990-2996.
Kilpatrick, D. R., C. F. Yang, et al. (2009). “Rapid group-, serotype-, and vaccine strain-specific identification of poliovirus isolates by real-time reverse transcription-PCR using degenerate primers and probes containing deoxyinosine residues.” J Clin Microbiol 47(6): 1939-1941.
La Monica, N., C. Meriam, et al. (1986). “Mapping of sequences required for mouse neurovirulence of poliovirus type 2 Lansing.” J Virol 57(2): 515-525.
Martin, J. and P. D. Minor (2002). “Characterization of CHAT and Cox type 1 live-attenuated poliovirus vaccine strains.” J Virol 76(11): 5339-5349.
Sambrook, J., E. F. Fritsch, et al. (1989). Molecular cloning. New York, Cold Spring Harbor Laboratory Press.
Stanway, G., P. J. Hughes, et al. (1984). “Comparison of the complete nucleotide sequences of the genomes of the neurovirulent poliovirus P3/Leon/37 and its attenuated Sabin vaccine derivative P3/Leon 12a1b.” Proc Natl Acad Sci USA 81(5): 1539-1543.
Sutter, R. W., T. J. John, et al. (2010). “Immunogenicity of bivalent types 1 and 3 oral poliovirus vaccine: a randomised, double-blind, controlled trial.” Lancet. 376(9753): 1682-1688.
van der Avoort, H. G., B. P. Hull, et al. (1995). “Comparative study of five methods for intratypic differentiation of polioviruses.” J Clin Microbiol 33(10): 2562-2566.
van Wezel, A. L. and A. G. Hazendonk (1979). “Intratypic serodifferentiation of poliomyelitis virus strains by strain-specific antisera.” Intervirology 11(1): 2-8.
van Wezel, A. L., J. A. van Herwaarden, et al. (1979). “Large-scale concentration and purification of virus suspension from microcarrier culture for the preparation of inactivated virus vaccines.” Dev Biol Stand 42: 65-69.
van Wezel, A. L., G. van Steenis, et al. (1978). “New approach to the production of concentrated and purified inactivated polio and rabies tissue culture vaccines.” Dev Biol Stand 41: 159-168.
Westdijk, J., D. Brugmans, et al. (2011). “Characterization and standardization of Sabin based inactivated polio vaccine: proposal for a new antigen unit for inactivated polio vaccines.” Vaccine 29(18): 3390-3397.
WHO (2003). Global Polio Eradication Initiative Strategic Plan 2004-2008. Geneva, WHO.

1-15. (canceled)

16. A method for the identification of a poliovirus strain in a sample, comprising selectively hybridising an oligonucleotide to the polioviral nucleic acid in the sample, wherein the oligonucleotide is at least one of:
a) an oligonucleotide comprising at least 12 contiguous nucleotides of SEQ ID NO: 1 or its complement and wherein the oligonucleotide comprises the sequence of positions 1942-1944 of SEQ ID NO: 1 or its complement at the 5′ or 3′ end;
b) an oligonucleotide comprising at least 12 contiguous nucleotides of SEQ ID NO: 1 or its complement and wherein the oligonucleotide comprises the sequence of positions 3894-3896 of SEQ ID NO: 1 or its complement at the 5′ or 3′ end;
c) an oligonucleotide comprising at least 12 contiguous nucleotides of SEQ ID NO: 2 or its complement and wherein the oligonucleotide comprises the sequence of positions 1942-1944 of SEQ ID NO: 2 or its complement at the 5′ or 3′ end; and,
d) an oligonucleotide comprising at least 12 contiguous nucleotides of SEQ ID NO: 2 or its complement and wherein the oligonucleotide comprises the sequence of positions 3894-3896 of SEQ ID NO: 2 or its complement at the 5′ or 3′ end;
whereby, selective hybridisation with the oligonucleotide in a) is indicative of the presence of a poliovirus strain selected from the group consisting of Mahoney type 1, Brunhilde, CHAT and Cox; selective hybridisation with the oligonucleotide in b) is indicative of the presence of the Mahoney type 1 poliovirus strain; selective hybridisation with the oligonucleotide in c) is indicative of the presence of the Sabin type 1 poliovirus strain; and, selective hybridisation with the oligonucleotide in d) is indicative of the presence of a poliovirus strain selected from the group consisting of Sabin type 1, CHAT and Cox.

17. The method according to claim 16, wherein the oligonucleotide comprises a mismatch to both SEQ ID NO: 1 and 2, or their complements.

18. The method according to claim 17, wherein the mismatch is at positions 1940, 1946, 3892 or 3898 of SEQ ID NO: 1.

19. The method according to claim 16, further comprising detecting the selective hybridisation of the oligonucleotide by an amplification or an amplification-ligation assay.

20. The method according to claim 19, comprising:
a) amplifying at least a portion of polioviral nucleic acid in the sample with a primer pair comprising a forward primer that is at least one of:
(i) a forward primer comprising at least 12 contiguous nucleotides and the 3′-end of the sequence: 5′-CCCTTTGACTTAAGTHCCAC-3′, wherein H is a nucleotide that is incapable of base pairing with C;
(ii) a forward primer comprising at least 12 contiguous nucleotides and the 3′-end of the sequence: 5′-CCCTTTGACTTAAGTHCAAA-3′, wherein H is a nucleotide that is incapable of base pairing with C;
(iii) a forward primer comprising at least 12 contiguous nucleotides and the 3′-end of the sequence: 5′-CCATGGTGTTCTTTTVTGTG-3′, wherein V is a nucleotide that is incapable of base pairing with A;
(iv) a forward primer comprising at least 12 contiguous nucleotides and the 3′-end of the sequence: 5′-CCATGGTGTTCTTTTVTTTT-3′, wherein V is a nucleotide that is incapable of base pairing with A;
(v) a forward primer comprising at least 12 contiguous nucleotides and the 3′-end of the sequence: 5′-GATTTACTCAGCAGAVTAGC-3′, wherein V is a nucleotide that is incapable of base pairing with A;
(vi) a forward primer comprising at least 12 contiguous nucleotides and the 3′-end of the sequence: 5′-GATTTACTCAGCAGAVTGGA-3′, wherein V is a nucleotide that is incapable of base pairing with A;
(vii) a forward primer comprising at least 12 contiguous nucleotides and the 3′-end of the sequence: 5′-AACTCTGTTATTTTGVCGCT-3′, wherein V is a nucleotide that is incapable of base pairing with A; and,
(viii) a forward primer comprising at least 12 contiguous nucleotides and the 3′-end of the sequence: 5′-AACTCTGTTATTTTGVCTCC-3′, wherein V is a nucleotide that is incapable of base pairing with A;
and a reverse primer, whereby a reverse primer in a pair with a forward primer produces an amplicon with the forward primers (i), (iii), (v) and (vii) on a reference cDNA template comprising the sequence of a Mahoney poliovirus strain or with the forward primer (ii), (iv), (vi) and (viii) on a reference cDNA template comprising the sequence of a Sabin type 1 poliovirus strain; and,
b) detecting whether an amplicon is obtained in step a), whereby an amplicon produced with at least one of forward primers (i), (iii), (v) and (vii) is indicative of the presence of a poliovirus strain selected from the group consisting of Mahoney, Brunhilde, CHAT and Cox; and, whereby an amplicon produced with at least one of forward primer (ii) (iv) (vi) and (viii) is indicative of the presence of the Sabin type 1 poliovirus strain.

21. The method according to claim 19, comprising:
d) amplifying at least a portion of polioviral nucleic acid in the sample with a primer pair comprising a forward primer that is at least one of:
(ix) a forward primer comprising at least 12 contiguous nucleotides and the 3′-end of the sequence: 5′-GATTTACTCAGCAGATAGGG-3′ (SEQ ID NO: 32);
(x) a forward primer comprising at least 12 contiguous nucleotides and the 3′-end of the sequence: 5′-AACTCTGTTATTTTGVCCCC-3′ (SEQ ID NO: 33), wherein V is a nucleotide that is incapable of base pairing with A;
and a reverse primer, whereby a reverse primer in a pair with a forward primer produces an amplicon with the forward primers (ix) and (x) on a reference cDNA template comprising the sequence of the Brunhilde poliovirus strain; and,
e), detecting whether an amplicon is obtained in step d), whereby an amplicon produced in step d) is indicative of the presence of the Brunhilde poliovirus strain.

22. The method according to claim 20, wherein the reverse primer comprises at its 3′-end a sequence of at least 14 contiguous nucleotides that are complementary to a sequence in an elongation product obtained on a polioviral template with a forward primer defined in claim 20.

23. The method according to claim 20, wherein the forward primer that is at least one of:
(i) a forward primer comprising the sequence:
5′-CCCTTTGACTTAAGTHCCAC-3′, wherein H is A, C, T or U; and,
(ii) a forward primer comprising the sequence:
5′-CCCTTTGACTTAAGTHCAAA-3′, wherein H is A, C, T or U;
and wherein the reverse primer is 5′-GATCCTGCCCAGTGTGTGTAG-3′.

24. The method according to claim 16, further comprising selectively hybridising an oligonucleotide to a polioviral nucleic acid in the sample, whereby the oligonucleotide is selective for one or more poliovirus strains selected from the group consisting of: the MEF-1 type 2 strain or the Lansing strain, the Sabin type 2 strain, the Saukett H or G strains, and the Sabin type 3 or the Leon strains.

25. The method according to claim 24, wherein the method comprises the steps of:
a) amplifying at least a portion of polioviral nucleic acid in the sample with a primer pair that is specific for one or more poliovirus strains selected from the group consisting of: the MEF-1 type 2 strain or the Lansing strain, the Sabin type 2 strain, the Saukett H or G strains, and the Sabin type 3 or the Leon strains; and,
b) detecting whether an amplicon is obtained in step a), whereby an amplicon produced with the primer pair specific for one or more of the poliovirus strains is indicative for the presence of those poliovirus strains.

26. The method according to claim 25, wherein in step a) the portion of polioviral nucleic acid is amplified with at least one primer pair selected from the group consisting of:
I) a forward primer comprising at least 12 contiguous nucleotides and the 3′-end of the sequence GGTTGTTGAGGGAGTCACGAGA and a reverse primer comprising at least 12 contiguous nucleotides and the 3′-end of the sequence CCCTGTCTCTACGGCTGTTAGC;
II) a forward primer comprising at least 12 contiguous nucleotides and the 3′-end of the sequence GCAATTACGCCGCAAGC and a reverse primer comprising at least 12 contiguous nucleotides and the 3′-end of the sequence GTGTAGGTGCTCCTGGAGGT;
III) a forward primer comprising at least 12 contiguous nucleotides and the 3′-end of the sequence AAGGAATTGGTGACATGATTGAGG and a reverse primer comprising at least 12 contiguous nucleotides and the 3′-end of the sequence CTCGGCTTTGTGTCAGGC; and,
IV) a forward primer comprising at least 12 contiguous nucleotides and the 3′-end of the sequence AATGACCAGATTGGTGATTCCTTG and a reverse primer comprising at least 12 contiguous nucleotides and the 3′-end of the sequence GTAAATGCGGACTTTGGAGGTTACT;
and whereby in step b) an amplicon produced with the primer pair in I) is indicative of the presence of the MEF-1 type 2 strain or the Lansing strain; an amplicon produced with the primer pair in II) is indicative of the presence of the Sabin type 2 strain; an amplicon produced with the primer pair in III) is indicative of the presence of the Saukett H or G strains; and an amplicon produced with the primer pair in IV) is indicative of the presence of the Sabin type 3 or the Leon strains.

27. The method according to claim 20, wherein an amplicon is detected by hybridisation with a fluorescent or chemiluminescent probe comprising a sequence that is complementary to a sequence in the amplicon.

28. The method according to claim 27, wherein the detection is in real time.

29. The method according claim 16, further comprising purifying RNA of the poliovirus in the sample and/or reverse transcribing the polioviral RNA to provide a polioviral cDNA.

30. An oligonucleotide, primer or probe selected from the group consisting of:
a) an oligonucleotide comprising at least 12 contiguous nucleotides of SEQ ID NO: 1 or its complement and wherein the oligonucleotide comprises the sequence of positions 1942-1944 of SEQ ID NO: 1 or its complement at the 5′ or 3′ end;
b) an oligonucleotide comprising at least 12 contiguous nucleotides of SEQ ID NO: 1 or its complement and wherein the oligonucleotide comprises the sequence of positions 3894-3896 of SEQ ID NO: 1 or its complement at the 5′ or 3′ end;
c) an oligonucleotide comprising at least 12 contiguous nucleotides of SEQ ID NO: 2 or its complement and wherein the oligonucleotide comprises the sequence of positions 1942-1944 of SEQ ID NO: 2 or its complement at the 5′ or 3′ end; and
d) an oligonucleotide comprising at least 12 contiguous nucleotides of SEQ ID NO: 2 or its complement and wherein the oligonucleotide comprises the sequence of positions 3894-3896 of SEQ ID NO: 2 or its complement at the 5′ or 3′ end.

31. A kit comprising:
a) at least one of an oligonucleotide, primer or probes selected from the group consisting of:
a) an oligonucleotide comprising at least 12 contiguous nucleotides of SEQ ID NO: 1 or its complement and wherein the oligonucleotide comprises the sequence of positions 1942-1944 of SEQ ID NO: 1 or its complement at the 5′ or 3′ end;
b) an oligonucleotide comprising at least 12 contiguous nucleotides of SEQ ID NO: 1 or its complement and wherein the oligonucleotide comprises the sequence of positions 3894-3896 of SEQ ID NO: 1 or its complement at the 5′ or 3′ end;
c) an oligonucleotide comprising at least 12 contiguous nucleotides of SEQ ID NO: 2 or its complement and wherein the oligonucleotide comprises the sequence of positions 1942-1944 of SEQ ID NO: 2 or its complement at the 5′ or 3′ end; and
d) an oligonucleotide comprising at least 12 contiguous nucleotides of SEQ ID NO: 2 or its complement and wherein the oligonucleotide comprises the sequence of positions 3894-3896 of SEQ ID NO: 2 or its complement at the 5′ or 3′ end; and
b) at least one of an enzyme and a buffer.
